(19) United States
(12) Patent Application Publication
(10) Pub. No.: US 2022/0053292 A1
(43) Pub. Date: Feb. 17, 2022

(54) Low Cost, High Performance Asset Tracking Systems and Methods

(71) Applicant: Patti Engineering, Inc., Auburn Hills, MI (US)

(72) Inventors: Samuel Mark Hoff, Clarkston, MI (US); Larry John Shipley, Upland, IN (US); Bradley Grabow, Auburn Hills, MI (US); Constance Amber Sadro, Metamora, MI (US)

(73) Assignee: Patti Engineering, Inc., Auburn Hills, MI (US)

(21) Appl. No.: 17/404,643

(22) Filed: Aug. 17, 2021

Related U.S. Application Data
(60) Provisional application No. 63/066,737, filed on Aug. 17, 2020.

(51) Int. Cl. H04W 4/029; H04W 48/20

(52) U.S. Cl. CPC H04W 4/029; G16H 50/80; H04W 48/20

(57) ABSTRACT

A real time asset tracking system and method is described including a plurality of mobile units (e.g., fobs) which are capable of communication with a network. The plurality of mobile units being configured to determine the presence of a contact event, e.g., when a first mobile unit is within a predetermined distance from a second mobile unit for a predetermined amount of time. If the first mobile unit is in a contact event, then the first mobile unit may be configured to alert the user (visual, vibration, sound) and report the contact event data to the server.

### REFERENCE TO RELATED PATENT APPLICATION

The present application claims benefit of U.S. provisional application No. 63/066,737, filed on Aug. 17, 2020.

### TECHNICAL FIELD

The subject matter described herein relates to systems and methods in the field of data analytics, including user location, asset location, and positioning and related navigation and analytics for the purpose of contact tracing, management of occupancy and congestion, wayfinding navigation, condition monitoring, and distance/flow studies.

### BACKGROUND

There are numerous benefits to asset tracking which are applicable in various industries and applications. For example, asset tracking can be used for traditional asset management and logistics or in any application where precision time and position tracking can be beneficial.

In one application, such as in a contagion-pandemic or post-pandemic era, there is an increasing need for businesses (e.g., restaurants, retail shops, manufacturing shop floors, etc.) to put in place a system that ensures customer and worker safety to reduce or eliminate downtime otherwise resulting from contaminated persons or assets which can be addressed with an asset tracking system. For example, precise asset tracking can be used for contact tracing and control and/or limit interactions within certain distances.

In another application, asset tracking can be used to enhance safety with regards to mobile machinery. For example, asset tracking may be used on loading docks to monitor the locations of individuals and mobile machinery (e.g., forklifts, transport vehicles, and robots) to alert the driver/vehicle if a contact event (e.g., collision) with an individual (or object) is likely or imminent.

In other application, precise asset tracking can be used in manufacturing facilities to coordinate the manufacture, assembly, machining, inventory, tracking, and delivery of items or parts on an efficient timeline.

### SUMMARY

One problem with existing people and/or asset tracking systems is that these systems are overly restrictive and passive, which results in wider contamination and longer shutdown periods and does not provide decision makers with information needed to make remedial workplace design measures. Plus, systems restrictions caused by the environment, such as limitation on wireless communications, privacy, and security, create specific problems that the systems and methods described herein are designed to overcome.

The present invention seeks to overcome these problems by, among other things, implementing an asset tracking system compatible with existing infrastructure and modularly designed that is capable of precise tracking of assets using mobile units that communicate with other mobile units using wireless communication to precisely determine the locations and positional relationships such as ultrawide band communications.

Further, to accommodate environments with varying levels of security restrictions and wireless data communications access concerns and restrictions (e.g., such as automobile assembly plants, LED manufacturing facilities, etc.), the mobile units may be configured to store the data regarding interactions with other mobile units and upload the stored data to the network only when in communication range with certain network access points (e.g., at a predetermined time and/or location).

The present system and method also include strategies to maximize accuracy and battery usage while maintaining data integrity and precision.

In certain embodiments, the present system and method is also designed, for example, to harvest real time data from the workspace, such as positional data of users in a workplace, which allows for remote troubleshooting, condition monitoring (e.g., two users within a predetermined distance for more than a predetermined period), and improved safety (e.g., via contact tracing and distance/flow studies).

### BRIEF DESCRIPTION OF DRAWINGS

The accompanying drawings constitute a part of this specification and illustrate embodiments that, together with the specification, explain the subject matter.

FIG. 1 shows various components of one embodiment of an asset tracking system.

FIG. 2 shows a flowchart of exemplary operation modes of mobile units.

FIG. 3 shows a flowchart of exemplary detection and analysis performed by mobile units in a comparison loop.

FIG. 4 shows an outline of exemplary features and related components of a mobile unit.

FIG. 5 shows an exemplary embodiment of a mobile unit.

FIG. 6 shows a flowchart of one embodiment of detection and analysis performed by asset tracking system including an anchor.

FIG. 7 shows a network diagram of one embodiment of an asset tracking system including an array of sensors used to capture environmental and/or system data.

FIG. 8 shows a flowchart of exemplary operation modes of an embodiment incorporating an array of sensors.

FIG. 9 shows a network diagram of one embodiment of an asset tracking system utilized in a dock safety application.

FIGS. 10A-C shows a flowchart of exemplary detection and analysis performed by an asset tracking system in a dock safety application at different events points within the process.

FIG. 11 shows a network diagram of one embodiment of an asset tracking system utilized in tracking environmental collection data.

FIG. 12 shows a flowchart of environmental collector application performed by the server, anchors, and mobile units at different events points within the process.

FIG. 13 shows a network diagram of one embodiment utilized in an emergency preparedness and reaction tool application.

FIG. 14 shows a flowchart of exemplary emergency preparedness application performed by the server, anchors, and mobile units at different events points within the process.

FIGS. 15-16 shows network diagrams of one embodiment utilized in a robot proximity monitoring application.

FIGS. 17A-E show a flowchart of exemplary robot safety application performed by the server (expanded in FIG. 17B), anchors (expanded in FIG. 17C), robot-mounted anchors (expanded in FIG. 17D), and mobile units (expanded in FIG. 17E) at different events points within the process.

### DETAILED DESCRIPTION

The following detailed description is provided with reference to the figures. Exemplary embodiments are described to illustrate the disclosure, not to limit its scope, which is defined by the claims. Those of ordinary skill in the art will recognize equivalent variations in the description that follows without departing from the scope and spirit of the disclosure.

Identically numbered reference characters correspond to each other so that a duplicative description of each reference character in the drawings may be omitted.

Below described are a variety of embodiments. Various components and features of each embodiment, however, may be incorporated into a variety of systems based on need including to combine features from multiple embodiments.

As will be used through this embodiment and others, the terms “user” and “asset” may be used interchangeable as the systems can be effectively utilized regardless whether the tracking is of a user, equipment, and/or other assets. For similar reasons “asset tracking” and “contact tracing” may also be used interchangeably.

### Embodiment 1

In a first embodiment, a real time location system (RTLS, system) 100 may be used for tracking assets, and/or users, in a variety of environments using, for example, a plurality of mobile units 10 carried individually by the users/assets desired to be monitored in a target environment.

In certain embodiments, the system 100 utilizes ultra-wideband (“UWB”) communication (described below) as a proximity measurement technology using active transponder technology that emits signals that only communicate with a stationary gateway (e.g., anchor 40). The active transponders do not necessarily interact with each other. The mobile units 10 interact with one another and with other system devices such as stationary anchors 40. The ability of the mobile units 10 to interact with each other allows the possibility for localized interactions that do not require a central controller and allows for data redundancy, for example, as a plurality of mobile units 10 capture interaction data for a single event.

Further, where typical RTLS systems require gateways to communicate transponder data to a central control system via a dedicated LAN connection, the mobile unit transponders in the system 100 communicate data to servers via existing network access points 20 (e.g., Wi-Fi gateway). If the network access point 20 is not accessible (e.g., too far away, no power, etc.), then the mobile unit 10 is configured to store interaction data until the mobile unit 10 is back within range of the network access point 20. Once back within range, the mobile unit 10 is configured to send the stored data to a server(s) 30.

According to one embodiment, the server 30 is comprised of two main parts: back end portion 31 and front end portion 32. According to this embodiment, for example, the back end portion 31 is designed to focus on the mobile unit 10 control, including managing the data uplink from the mobile units 10 and mobile unit 10 firmware updates. The back end portion 31 may also be configured to perform any triangulation of position. Data from the mobile units 10 may also be stored in the back end portion 31 database for use of the front end portion 32 of the server 30. As described below, the front end portion 32 is where user experience is presented such as dashboards, reports display and outputs, and any high level configurations.

The system 100 may utilize anchors 40 as reference positions and to improve positional location measurement precision. In certain embodiments, the anchors 40 may be a stationary version of a mobile unit 10 and may include additional features such as functioning as additional access points 20 with connections to the network and/or server 30.

One function of the anchors 40 is to give a reference point to the mobile units 10 for location purposes. The anchors 40 are not necessarily configured to be used as additional access points 20, however, may optionally be designed to function as access points 20. According to one embodiment, for example, the anchors 40 may be arranged 20′ apart from each other inside a facility. This aspect of the system 100 is described in further detail below.

The system 100 designed with the anchors 40 is configured to utilize network access points 20 to connect to the server 30 via a user's existing network infrastructure, whereas typical RTLS gateways require a LAN connection, usually continual, back to a central control system (e.g., which is less flexible and more expensive). The anchors 40 may be hardwired for power rather than use batteries and may have fixed coordinates. The software on the server 30 is configured to determine mobile unit 10 locations by triangulating their positions from the anchor 40 measurements.

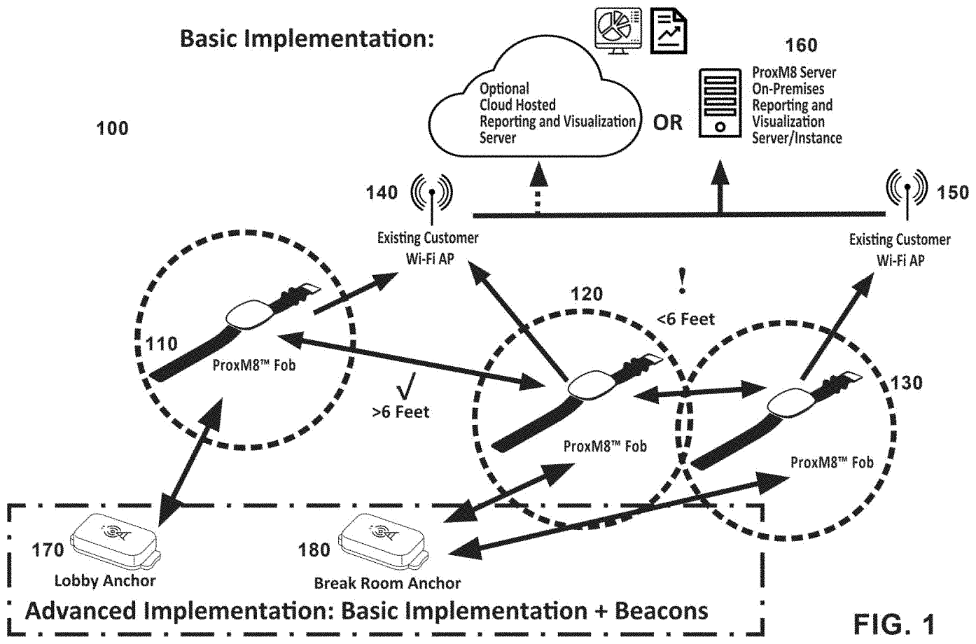

As shown in FIG. 1, one embodiment of the asset tracking system 100 comprises at least one mobile unit 10, which may also be referred to as electronic data carriers, wearables, FOBS, lanyards, clip-ons, bracelets, or similar.

The mobile unit 10 may be carried by an asset (e.g., “user”) in a variety of environments. The mobile unit 10 is capable of, for example, (i) determining mobile unit data such as distances and durations of interactions with other mobile units 10, (ii) storing the mobile unit data, (iii) sending the mobile unit data to the server 30, (iv) obtaining updates via the access points 20, and (v) providing feedback such as visual (e.g., LED lights), physical (e.g., haptic), and/or audial (e.g., beeps).

The data collected by the mobile units 10 (e.g., mobile unit data) may include contact event data, namely start time, end time, minimum distance detected between mobile units 10, top 5 (more or less can be collected depending on requirements) closest access points 20 (BSSIDS) and closest anchor 40 (if applicable).

The mobile units 10 may use wireless technology such as Bluetooth, WiFi, or ultra-wideband (UWB) communication (discussed below) to determine the mobile unit data, e.g., measure distances between mobile units 10.

In one embodiment, the mobile units 10 utilize UWB communications to determine distances between mobile units 10 to more precisely determine distances without incurring data errors or inconsistencies present with other technologies. UWB is a radio technology that can use a very low energy level for short-range, high-bandwidth communications over a large portion of the radio spectrum. One advantage of UWB is that it minimizes distance errors incurred due to the directionality of the signals or directionality between the mobile units 10.

In addition to improved positional accuracy, UWB positioning may also be desirable in environments with limited network access due to limited connections and/or security restrictions because UWB can be transmitted and calculations can be performed using just the mobile units 10. Thus, the mobile units 10 do not need to be within range of the network such as used in traditional systems (e.g., Wi-Fi positioning) to obtain position data and consequently issue alerts.

In some embodiments, the mobile units 10 are capable of determining distance form other mobile units 10, but do not calculate their absolute positions. Conversely, in some embodiments, the mobile units 10 include a map representation of any fixed units (e.g., anchors 40) to be able to triangulate position.

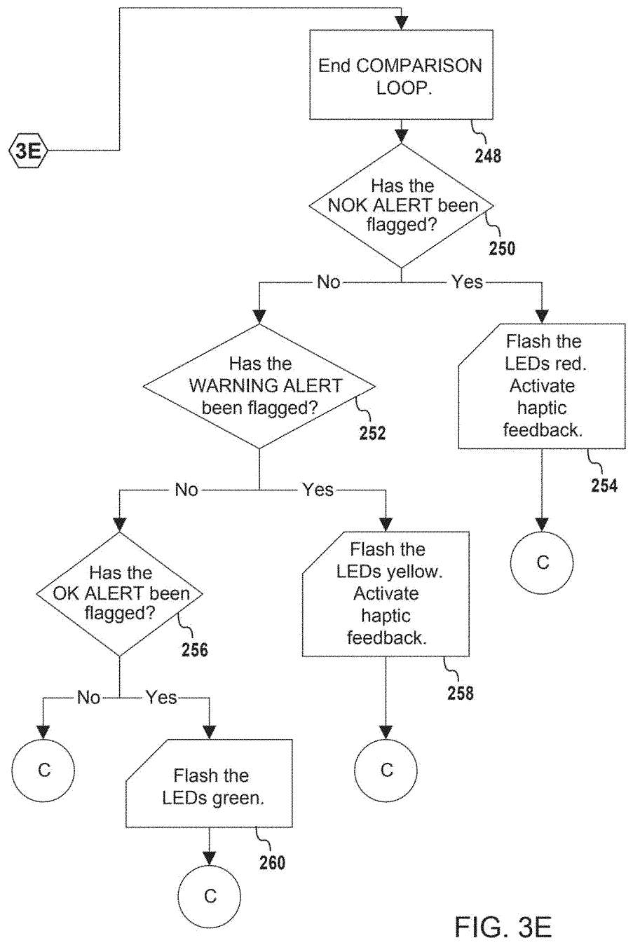

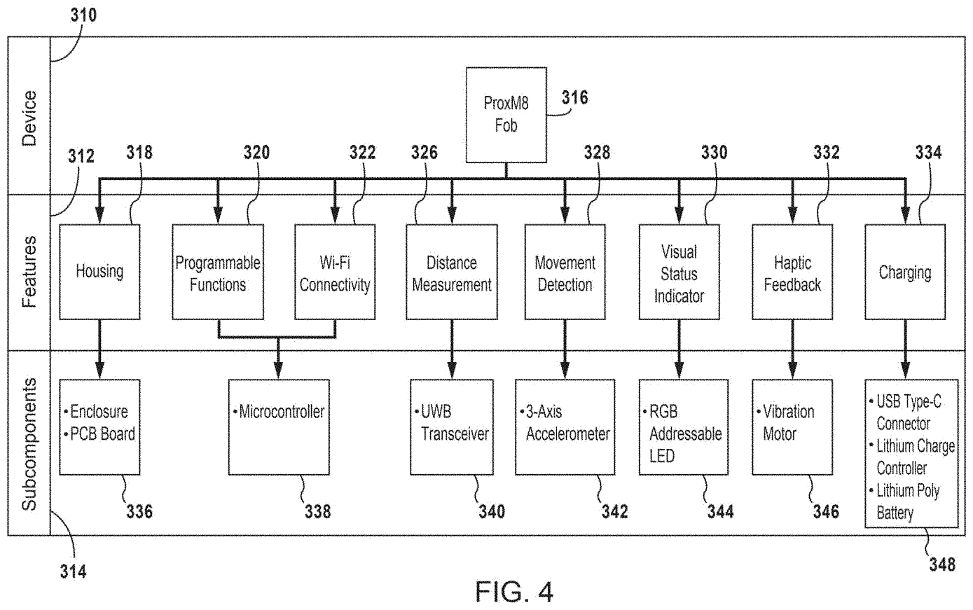

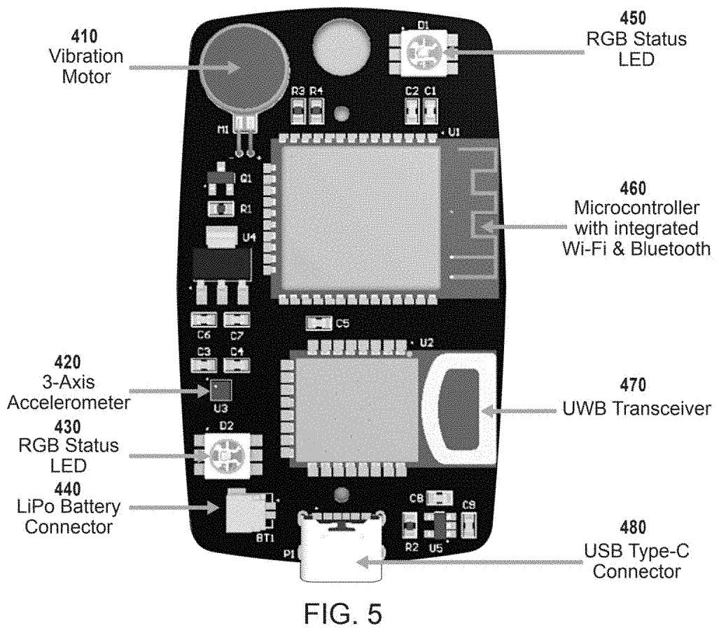

FIGS. 4 and 5 illustrate an exemplary embodiment of the mobile unit 10, including microcontroller 12 (e.g., with integrated Wi-Fi and Bluetooth™), accelerometer 13 (e.g., 3-axis accelerometer or proximity probe), at least one LED 14 (e.g., RGB status), vibration motor 15, charging port 16 (e.g., USB type-C connector), and UWB transceiver 17 positioned within housing 11. The mobile unit 10 may designed with some, all, or additional elements according to desired functionality of the mobile unit 10 and the system 100. The elements may be arranged in the manner shown in FIG. 5, or other configurations based on design choice and/or constraints.

The housing 11 is configured to enclose a PCB and other components of the mobile unit 10 shown in FIG. 5. The microcontroller 12 is configured to be programmable and enable Wi-Fi and/or Bluetooth™ and/or other wireless connectivity. The connectivity enables the mobile unit 10 to receive updates and submit data (e.g., EVENT LOGs) to the server 30 (described below) via the access point(s) 20. The UWB transceiver 17 (described above) is designed for distance measurement, e.g., measuring distance to other mobile units 10 in the system 100. The accelerometer 13 may be a 3-axis accelerometer designed to detect body movements and measure step count and distance travelled. One advantage is that the accelerometer 13 allows the mobile unit 10 to determine distance measurements in non-GPS enabled environments. The vibration motor 15 is configured to provide haptic feedback to the user/wearer, e.g., haptic feedback employs the use of a vibrating component or an actuator, such as eccentric rotating mass (ERM) motors and linear resonant actuators (LRA). The mobile unit 10 may also include a battery 18 to power the components, e.g., a USB type-c connector, lithium charge controller, and a lithium poly battery.

In one embodiment, the mobile unit 10 includes user notification/alert and/or feedback systems.

For example, the mobile units 10 may include visual status indicators, such as LEDs 14 or an electronic display (not shown). These visual status indicators can be designed to indicate various information such as remaining battery life or to alert the user to contact event information. For example, the visual indicators may alert the user/wearer (and others within visible range of the user/wearer) of the beginning/end of a preliminary contact event or contact event.

Similarly, the mobile units 10 may include a haptic feedback mechanism such as the vibration motor 15 to alert the user to the same, similar, or different information. The mobile units 10 may also include an auditory feedback mechanism (not shown) to alert the user to the same, similar, or different information.

The mobile units 10 may include external device control functionality to control the equipment such as emergency cut-off functionality in the case of mobile equipment. For example, in the case of a fork-lift in a contact or near-contact event, the system may be capable of equipment shutoff to prevent a collision or other damage.

The mobile units 10 may include a method of charging, such as USB Type 3, inductive charging, or other known charging methods. The embodiment shown in FIG. 5 include the charging port 16 and the battery 18.

The mobile units 10 may be worn by users as a fob, such as in a user's pocket or keychain, on a wristband, arm band, lanyard, or otherwise integrated into an electronic device such as a smart phone or smart watch. In the case of equipment, the mobile units 10 may also be hardwired into the equipment.

The mobile unit 10 may be used to track the movements of assets such as industrial equipment, goods, electronics, animals, or various types of assets.

The asset tracking system 100 may be designed to include one or more access points 20, e.g., device that allows wireless devices to connect to network. The access points 20 are connection points configured to communicate with the mobile units 10 and the server 30.

The access points 20 may be any type of access points (e.g., APs or WAPs) configurable to send and/or receive data from the mobile units 10 and communicate that data to the network 30. For example, the access points 20 may comprise local area network devices, such as Wi-Fi standard access points.

In another embodiment, the access points 20 may be removed or integrated into mobile units 10, such that mobile units 10 can communicate directly to the network 30.

The server 30 includes processing and access means of obtaining the data from the mobile units 10 and generating and displaying the information on a dashboard, accessible by one or more users (e.g., system administrator, manager, etc.). The server 30 may comprise typical known server components (e.g. networked computer components). The server 30 may be connected to the system 100 via a network and for purposes of this disclosure, server and network may be used interchangeably.

The dashboard may run on a mobile application (e.g., phone or tablet) and/or webpage on a computer.

The dashboard is designed to enable the user to input and/or define several system setup parameters for contact events such as (i) minimum distance that mobile units 10 must be in order to not register a contact event (DISTANCE THRESHOLD), (ii) minimum distance hysteresis used to end a contact event (SEPARATION DISTANCE) to ensure that the mobile unit software is using the hysteresis to not prematurely end a contact event and then immediately begin a new contact event should the mobile unit 10 be right on the minimum distance, and (iii) amount of time the mobile units 10 can be within the minimum distance before registering a contact event (DURATION THRESHOLD).

The dashboard may be designed to enable the user to setup associations between the mobile units 10 and the anchors 40 if applicable. For example, the user will be able to enter (symbolic) names/identities for each device (e.g. mobile unit 10 or anchor 40) to be able to read the reports more efficiently.

The dashboard may be designed to enable the user to view contact events and/or run reports. For example, the user will be able to view the details of a contact event (general location, time duration, distance, time stamp).

The dashboard may be designed to allow the user to run one or more reports with filters to analyze the data. For example, a user could filter based on a particular mobile unit 10 to trace the contacts of a person carrying the particular mobile unit 10 determined to have a condition the user desires to trace; filter by date to quantify the effectiveness of new or existing policies; or filter by location to determine whether certain locations require more policies.

As shown in FIG. 1, the server 30 may be hosted in a networked storage such as in the cloud or in an on-premises server, or combination thereof.

As shown in FIG. 1, in another embodiment (i.e., “Advanced Implementation”), the system 100 may further comprise one or more anchors 40, which also may be referred to as beacons. The anchors 40 are configured to communicate (send and receive data) with the mobile units 10 such that a position of the mobile units 10 can be more precisely and efficiently determined within a premises and/or the distance between the mobile units 10 can be more precisely or efficiently determined. More specifically, the anchors 40 may be stationary devices intended to give a reference point, for location purposes, to the mobile units 10.

The anchors 40 may be assigned (symbolic) names/identities to help the user contextualize and read the reports generated by the dashboard. For example, if a plurality of the mobile units 10 have a contact event within range of anchor 40A (defined as “Break Room Anchor”), then contact event data may be updated to include “Break Room Anchor”. Additionally, the contact data may include information on the closest anchor[s] 40 to the Break Room Anchor 40A.

Based on positional relationships of the mobile unit 10 to the anchor 40, for example, other mobile unit 10 data can be logged, e.g., such as length of time in proximity to an anchor 40. Furthermore, by knowing the fixed locations of the anchors 40, more precise location data concerning the location of the mobile unit 10 can be obtained.

The anchors 40 may be configured to connect to the server 30 to receive updates, but may or may not function as access points 20.

In the system shown in FIG. 1, the mobile units 10 are configured to locate themselves during a contact event. This can be accomplished, for example, by the mobile units 10 logging basic service set identifiers (BSSIDs) of the closest known access points 20 and/or anchors 40, described below.

For example, data related to preliminary contact events and contact events, “EVENT LOGs” is first stored locally on the mobile units 10. After a defined period referred to as a “TRANSMISSION THRESHOLD”, the mobile units 10 may connect to the server 30 via an access point 20 to upload the data.

The mobile unit 10 data collected includes, for example, contact event data such as start time, end time, minimum distance detected between mobile units, top 3-7, preferably 5 closest access points, closest anchor (if applicable).

Once the data is stored on the server 30, it will be accessible to the user via the dashboard described above.

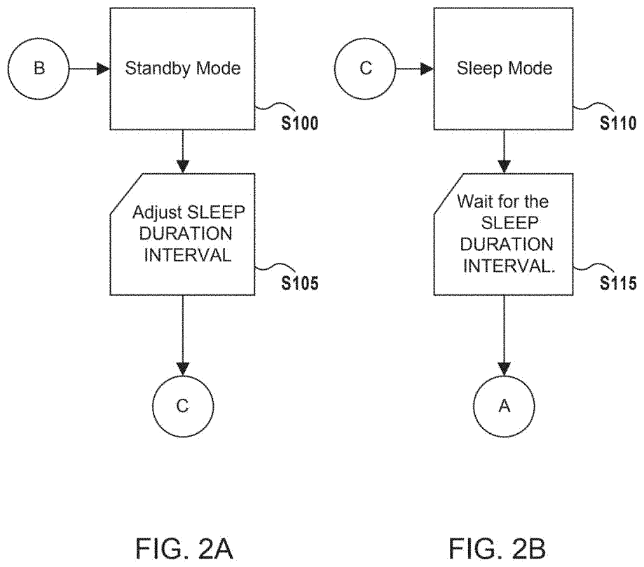

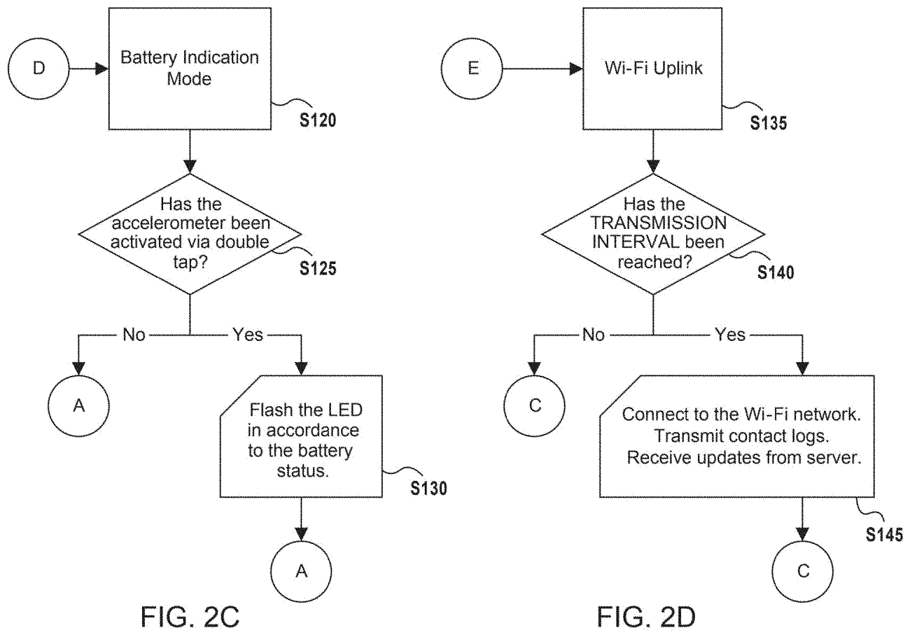

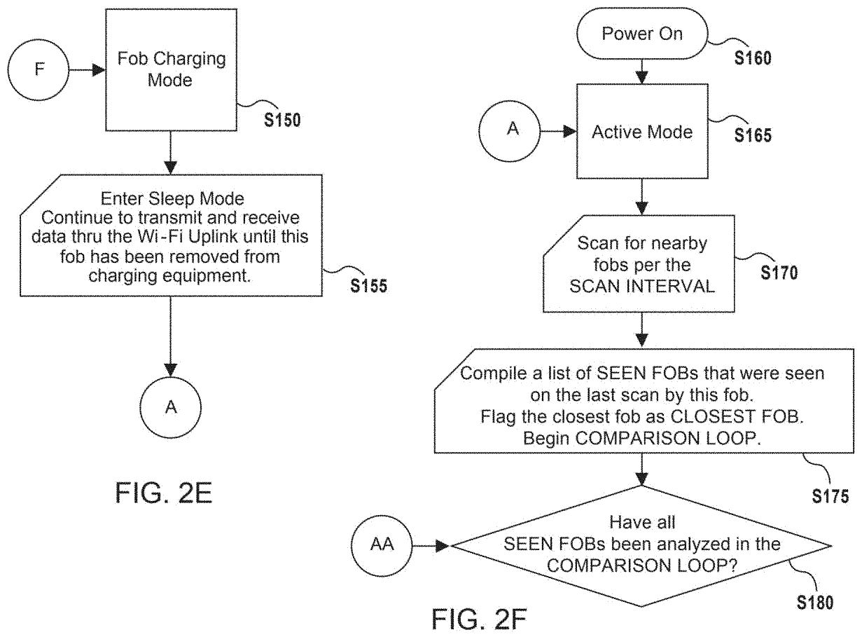

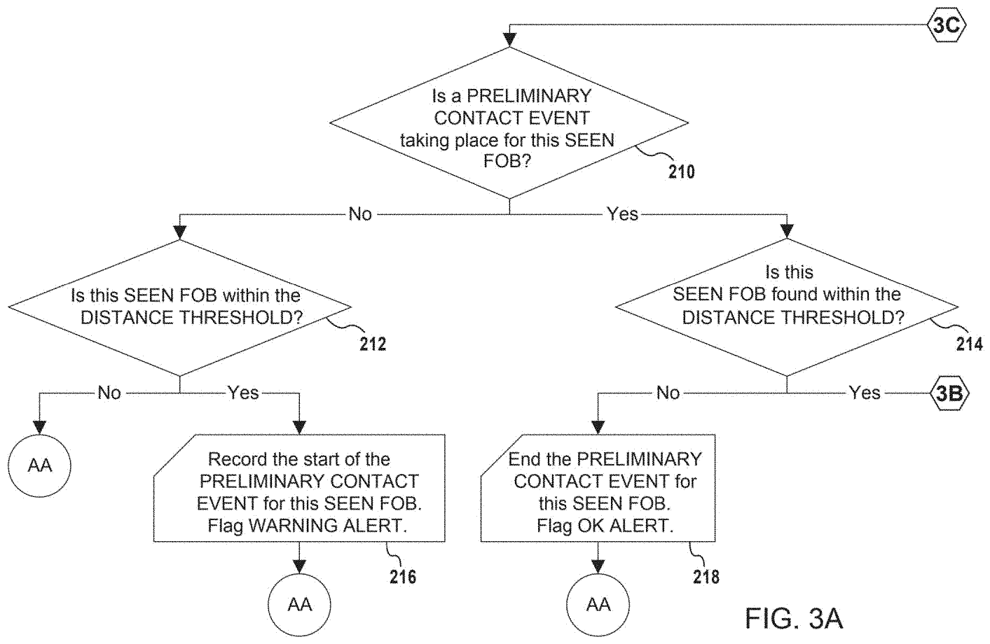

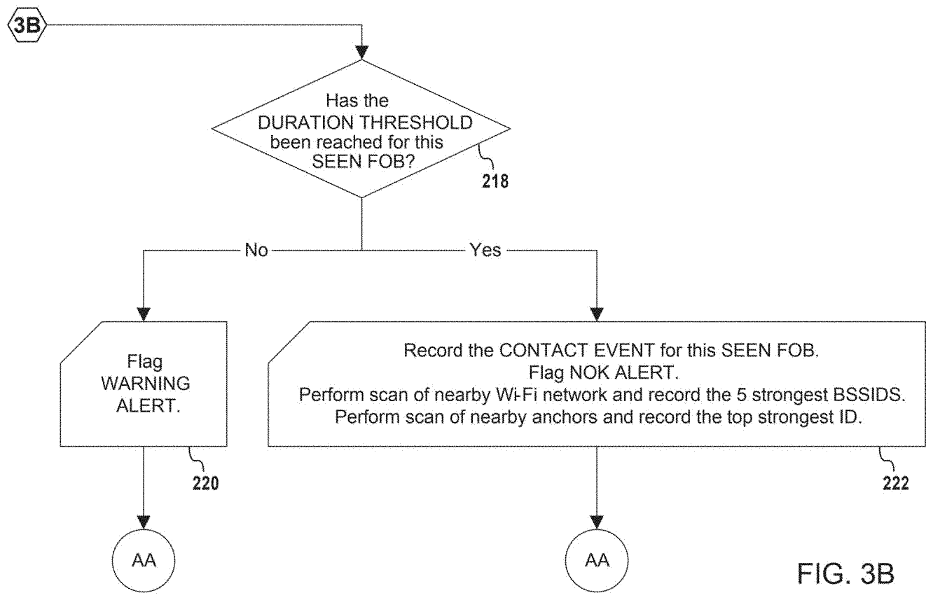

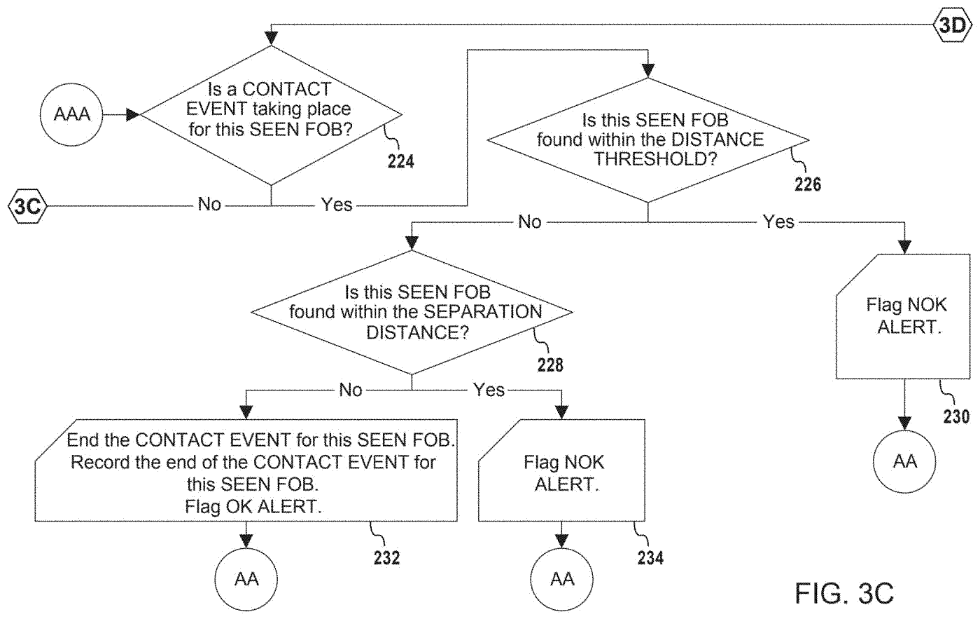

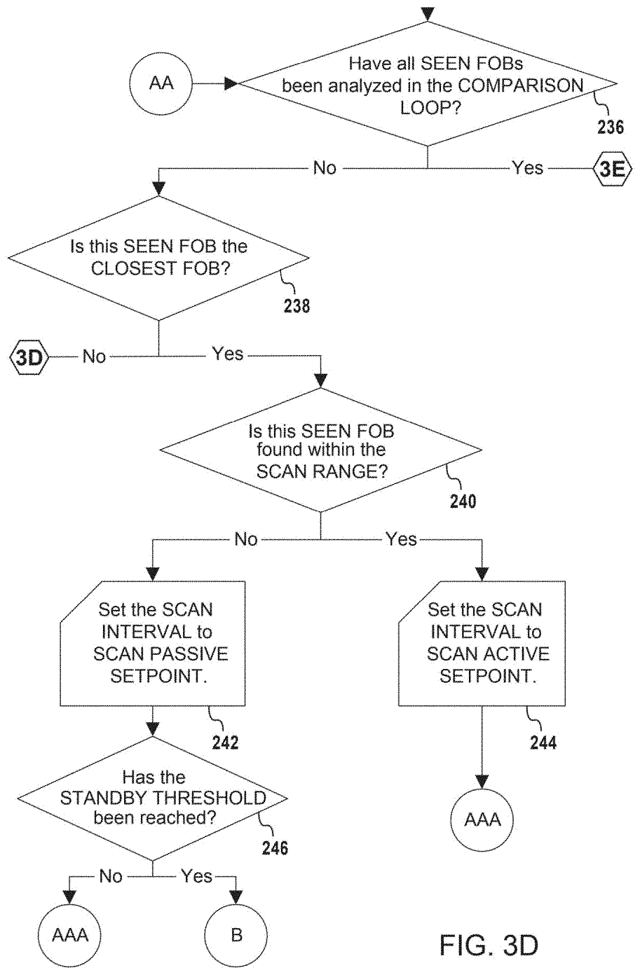

FIGS. 2-3 shows exemplary flowcharts of various operations of the mobile units 10 to further illustrate functionality described above.

As shown in FIG. 2, for example, there are multiple modes of operation of mobile unit 10, e.g., SLEEP MODE, BATTERY INDICATION MODE, UPLINK MODE, CHARGING MODE, and ACTIVE MODE. Each of these modes of operation is described below. However, it should be appreciated that other embodiments may comprise additional or alternative steps, or may omit one or more steps altogether. It should also be appreciated that other embodiments may perform certain steps in a different order; steps may also be performed simultaneously or near-simultaneously with one another.

In the SLEEP MODE, the mobile unit 10 enters a battery saving state for a period of time (SLEEP DURATION INTERVAL) and reduces the frequency of scans for other mobile units 10, anchors 40, and access points 20.

The SLEEP MODE serves as a method of conserving even more battery life when there are not any other mobile units 10 in the area. During the SLEEP MODE, the scan interval is extended to increase the amount of time spent sleeping. Every few cycles, the mobile unit 10 may listen for the full duration of the scan interval to guarantee interception of packets from other mobile units 10.

For example, every nth cycle, the mobile unit 10 may perform a full-length observation throughout the scan interval for other nearby mobile units 10 and every other cycle, listen for only a short period of time for other nearby mobile units 10. For example, the mobile units could perform at least every other (2) cycle and/or less than every fifth cycle though this number will depend on the SCAN INTERVAL DURATION. In some embodiments, the mobile units 10 are configured to contact other mobile units 10 (or other devices) at least once every two minutes or roughly thereabout. This duration would allow time for the mobile units 10 to wake up as two devices converge on each other's locations.

In BATTERY INDICATION MODE, the mobile unit 10 indicates the remaining battery life. This can occur, for example, after user initiation (e.g., after a user double taps the mobile unit).

In UPLINK MODE, the mobile unit 10 will periodically (TRANSMISSION INTERVAL) connect to the server 30 to transmit event logs and to receive updates from the server 30.

In CHARGING MODE, which occurs when the mobile unit 10 is charging, the mobile unit 10 will enter the SLEEP MODE and stop scanning for other mobile units 10. However, the mobile unit 10 may still connect to the server 30 to receive updates and transmit event logs (per the TRANSMISSION INTERVAL).

In ACTIVE MODE, the mobile unit 10 is configured to scan for other mobile units 10. After each scan, on-board software will run all the other known mobile units 10 (SEEN FOBs) found though a COMPARISON LOOP and categorize each mobile unit 10 into categories such as (a) safe mobile unit, (b) preliminary contact mobile unit, and/or (c) contact mobile unit.

The ACTIVE MODE also serves as a method of ensuring accurate and fast data collection at the expense of battery life. The ACTIVE MODE is primarily enabled when at least one other mobile unit 10 is within a specified distance (e.g., within 6 or 10 feet). During the ACTIVE MODE, the scan interval is framed such that receipt of packets from any nearby mobile unit 10 is guaranteed during every cycle. In this mode, for example, the mobile unit 10 ensures that it is listening for at least half of the scan interval cycle to confirm intercept from other nearby devices 10.

After all nearby mobile units 10 (SEEN FOBs) have been categorized, the user (e.g., wearer of a mobile unit 10) can be alerted using the status indicators described herein. The alerts are prioritized, and the on-board software may be configured to only activate one alert at a time.

For example, the NOK ALERT may be indicated, e.g., with red LED and/or haptic feedback, to indicate to the user that the user is in a “not okay” condition. For example, the not okay condition could be defined as the user is in a contact event and should take action to end the condition such as by moving further from the other mobile unit 10 user.

The WARNING ALERT may be indicated, e.g., with yellow LED and/or haptic feedback, to indicate that the user is in a warning condition. For example, the warning condition could be defined as the user is in a preliminary contact event and should take action to end the condition such as by moving further from the other user.

The OK ALERT may be indicated, e.g., with green LED, to indicate that the user is in an okay condition. For example, the user has ended a PRELIMINARY CONTACT EVENT or CONTACT EVENT and is at a safe distance (e.g., more than 6 feet) from other mobile units 10.

FIG. 3 shows a flowchart of detection and analysis performed by the mobile units 10 in the COMPARISON LOOP. However, it should be appreciated that other embodiments may comprise additional or alternative steps, or may omit one or more steps altogether. It should also be appreciated that other embodiments may perform certain steps in a different order; steps may also be performed simultaneously or near-simultaneously with one another.

First, the mobile unit 10 determines whether all SEEN FOBs (e.g., mobile units 10) have been analyzed in the COMPARISON LOOP.

If all SEEN FOBs have been analyzed, then the mobile unit 10 determines whether a NOK ALERT has been flagged. If yes, then the mobile unit 10 indicates the NOK ALERT, e.g., with red LED and/or haptic feedback, and then enters the SLEEP MODE.

If the NOK ALERT has not been flagged, then the mobile unit 10 determines whether the WARNING ALERT has been flagged. If yes, then the mobile unit 10 indicates the WARNING ALERT, e.g., with yellow LED and/or haptic feedback, and then enters the SLEEP MODE. If no, then the system 100 determines whether the OK ALERT has been flagged. If the OK ALERT has been flagged, then the mobile unit 10 indicates the OK ALERT, e.g., with green LED, and then the mobile unit enters SLEEP MODE. If the OK ALERT has not been flagged, then the mobile unit 10 enters the SLEEP MODE.

If all SEEN FOBS have not been analyzed in the COMPARISON LOOP, then the system 100 determines whether the next SEEN FOB is the closest mobile unit 10.

If the next SEEN FOB is the not the closest mobile unit 10, then the mobile unit 10 determines whether a CONTACT EVENT is taking place for the SEEN FOB.

If the next SEEN FOB is the closest mobile unit 10, then the mobile unit 10 determines whether the next SEEN FOB is within a SCAN RANGE. If yes, then the mobile unit 10 sets the SCAN INTERVAL to SCAN ACTIVE SETPOINT and then determines whether a CONTACT EVENT is taking place.

If SEEN FOB is not within SCAN RANGE, then the mobile unit sets the SCAN INTERVAL to scan passive setpoint and determines whether the standby threshold has been met. If the standby threshold has been met, then the mobile unit 10 enters standby mode.

The standby mode serves as a method of conserving battery life when other mobile units 10 are heard but beyond a specified significant distance (not within 10 feet). During the standby mode, the scan interval is extended to increase the amount of time spent sleeping. Every few cycles, the mobile unit 10 will listen for the full duration of the scan interval to guarantee interception of packets from other mobile units 10.

For example, every nth cycle, perform a full length observation throughout the scan interval for other nearby mobile units 10 and every other cycle, listen for only a short period of time for other nearby nodes.

If the standby threshold has not been met, the mobile unit 10 determines whether a CONTACT EVENT is taking place.

If a CONTACT EVENT is taking place, the mobile unit 10 determines whether the SEEN FOB is within the DISTANCE THRESHOLD. If yes, then the mobile unit 10 indicates a NOK ALERT and then reverts to the determination whether all SEEN FOBs have been analyzed in the Comparison Loop.

If the SEEN FOB is not within the distance threshold, then the mobile unit 10 determines whether the SEEN FOB is within the separation distance. If so, the mobile unit 10 indicates a NOK Alert and then reverts to the determination whether all SEEN FOBs have been analyzed in the COMPARISON LOOP.

If the SEEN FOB is not within the SEPARATION DISTANCE, then the mobile unit 10 ends the CONTACT EVENT for the SEEN FOB, records the CONTACT EVENT for the SEEN FOB, indicates the OK ALERT, and then reverts back to the determination whether all SEEN FOBs have been analyzed in the COMPARISON LOOP.

If a CONTACT EVENT is not taking place, then the mobile unit 10 determines whether a PRELIMINARY CONTACT EVENT is taking place for the SEEN FOB.

If a PRELIMINARY CONTACT EVEN is taking place for the SEEN FOB, then the mobile unit 10 determines whether the SEEN FOB is within the DISTANCE THRESHOLD.

If the SEEN FOB is within the DISTANCE THRESHOLD, then the mobile unit 10 determines whether the DURATION THRESHOLD has been reached by the SEEN FOB. If yes, then the mobile unit 10 records the CONTACT EVENT for the SEEN FOB, flags the NOK ALERT, scans the server 30 and records, for example, the five strongest BSSIDs, optionally scans the anchors 40 and records, for example, the five strongest, then reverts to the determination whether all SEEN FOBs have been analyzed in the COMPARISON LOOP.

If the SEEN FOB has not reached the DURATION THRESHOLD, then the mobile unit 10 flags the WARNING ALERT and reverts to the determination whether all SEEN FOBs have been analyzed in the COMPARISON LOOP.

If the SEEN FOB is not within the DISTANCE THRESHOLD, then the mobile unit 10 ends the PRELIMINARY CONTACT EVENT for the SEEN FOB, flags the OK Alert, and reverts to the determination whether all SEEN FOBs have been analyzed in the COMPARISON LOOP.

If a PRELIMINARY CONTACT EVENT is not taking place for the SEEN FOB, then the mobile unit 10 determines whether the SEEN FOB is within the DISTANCE THRESHOLD. If yes, then the mobile unit 10 records the start of the PRELIMINARY CONTACT EVENT for the SEEN FOB, flags the warning alert, and reverts to the determination whether all SEEN FOBs have been analyzed in the COMPARISON LOOP.

If the SEEN FOB is not within the DISTANCE THRESHOLD, then the mobile unit 10 reverts to the determination whether all SEEN FOBs have been analyzed in the COMPARISON LOOP.

The system 100 may also include geofencing to define a geographic boundary, e.g., a virtual barrier. This will allow the administrator (user) to set up triggers that send status indicators or other notifications to a user (wearer) when the mobile unit 10 enters or exits the specified area. The mobile unit 10 may also enter SLEEP MODE when exiting the specified area to conserve battery power.

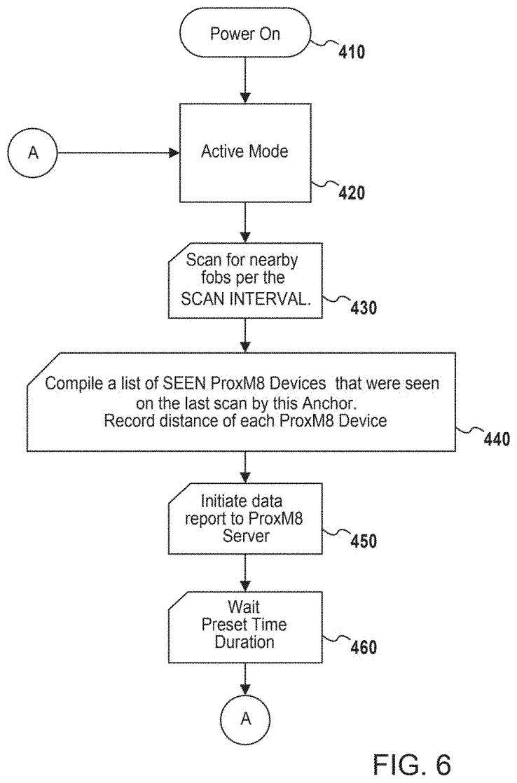

FIG. 6 shows a flowchart of one embodiment of detection and analysis performed by the asset tracking system 100 further including the anchor 40.

As shown in FIG. 6, when the anchor 40 is powered on, it enters ACTIVE MODE. Then, the anchor 40 scans for nearby mobile units 10 for the SCAN INTERVAL.

After scanning for nearby mobile units 10, the anchor 40 compiles a list of SEEN mobile units 10 obtained running the scan and records the distance to each SEEN mobile unit 10.

Subsequently, the anchor 40 initiates a data report to server 30 and either send the data directly to server 30, send the data to the server 30 via the access point 20, or sends the data to at least one mobile unit 10 to be transmitted to the server 30 by the mobile unit 10.

Then, the anchor 40 waits a PRESET DURATION TIME and then reverts to ACTIVE MODE and repeats the process.

As with previous embodiments, all the defined characteristics of the system for the anchors 40, such as the SCAN INTERVAL, can be set by the user, for example, vie the web dashboard.

### Embodiment 2

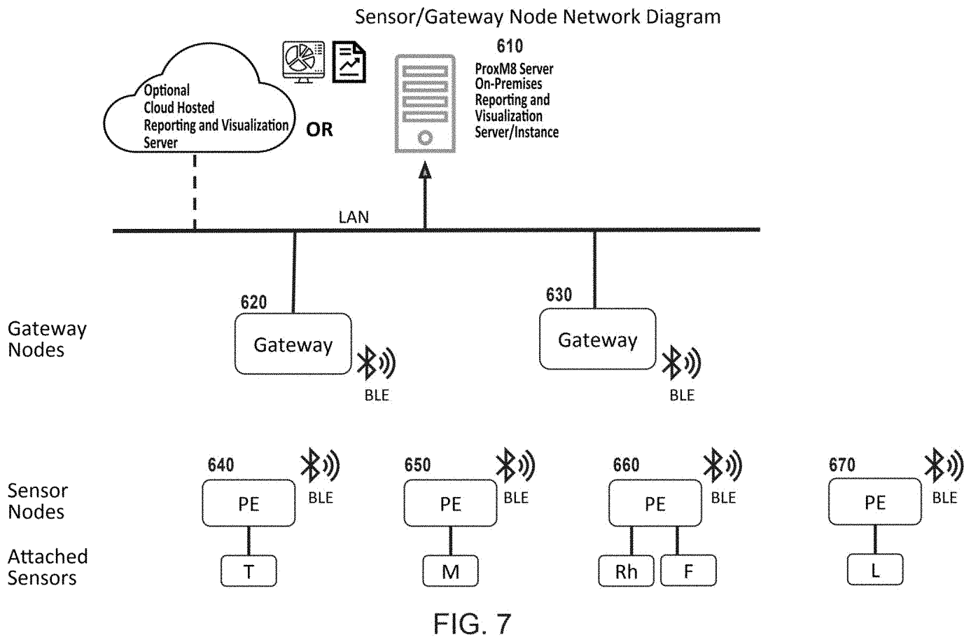

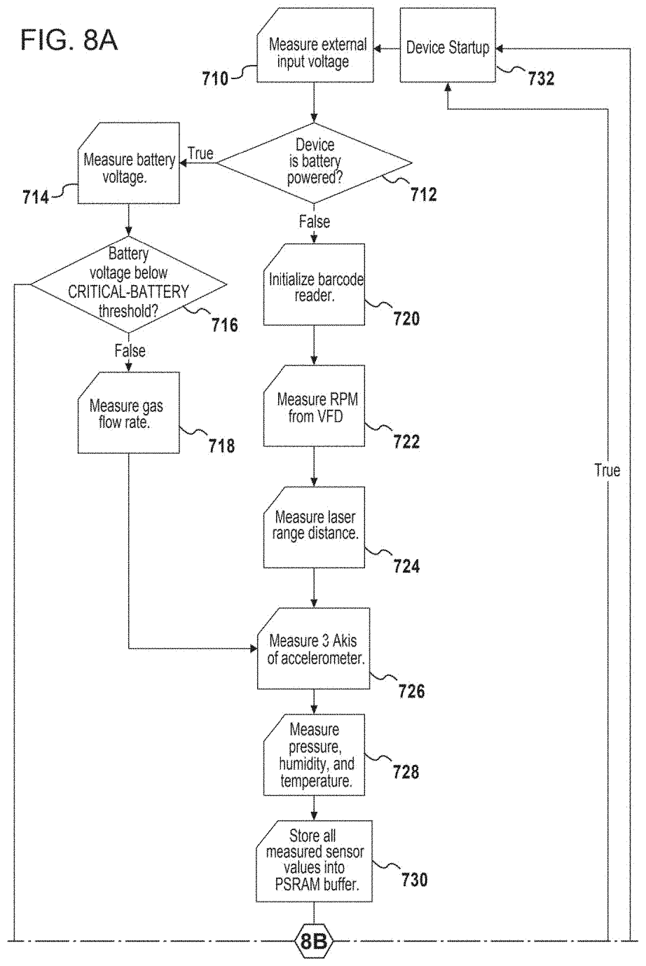

In another embodiment, as shown for example in FIG. 7, the asset tracking system contains an array of various sensors that are used to capture environmental and/or system data (temperature, humidity, pressure, flow rate, vibration—for environmental, logged barcode scans, vfd speeds, for system data).

As shown in FIG. 7, the system includes servers 730, which may also be referred to as network 730, communicating with gateway devices 710 which in turn communicate with sensor nodes 720 attached to various sensors 730 via communications such as Bluetooth. In such a system, the sensors 730 contain measurement data which is then sent from the attached sensor nodes 720 to the gateway devices 710 and the send to server 30. The sensors 730 may be fixed or may also be attached to mobile units 10.

The data can also be locally filtered and logged. Periodically, the nodes/mobile units 10 can also report the data back to a gateway device 710 wirelessly. If a gateway device 710 is not available, the data can be stored and then forwarded later.

The gateway device 710 are devices that wirelessly monitor and receive data from the sensor nodes 720 and the mobile units 10 in the field. The data can be received and stored locally and optionally shared (wirelessly or hard-wired) for long term storage, analytics, etc.

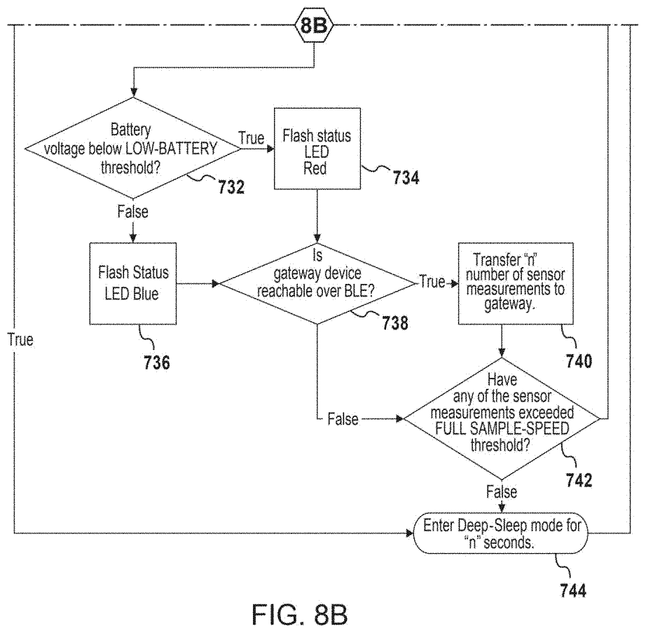

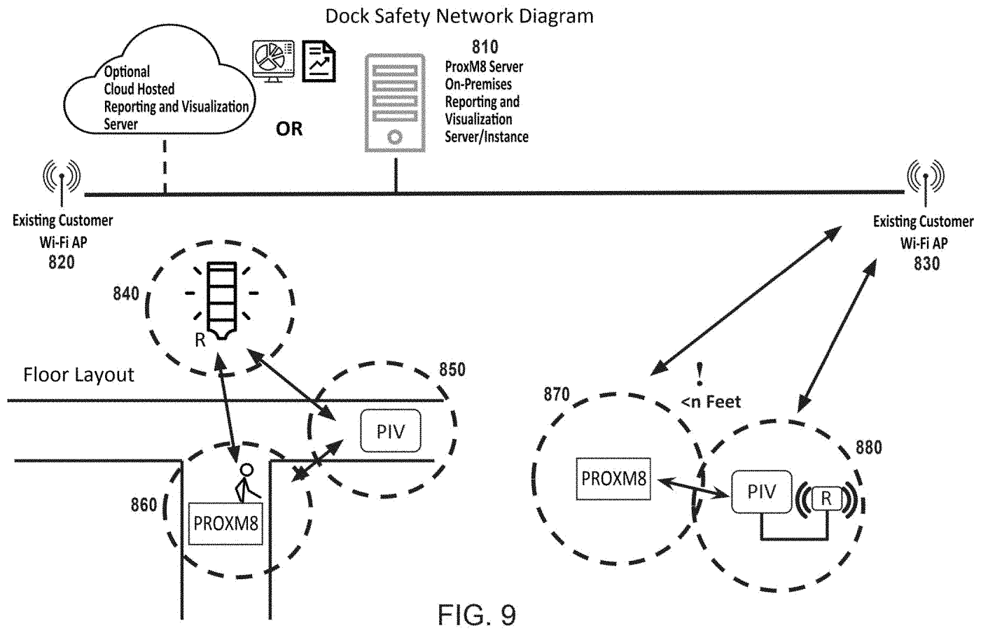

FIG. 8 shows a flowchart of exemplary operation modes of an embodiment with the above sensors.

At DEVICE STARTUP, the sensor node 720 measures external input voltage and determines whether the battery is powered

If the battery determination is true (e.g. SUFFICIENT BATTERY POWER is present), the sensor node 720 measure the battery voltage and determines whether the battery voltage is below CRITICAL BATTERY THRESHOLD.

If the battery voltage is below the CRITICAL BATTERY THRESHOLD, then the sensor nodes 720 enters DEEP-SLEEP MODE for a PREDETERMINED SLEEP TIME (e.g., “n” seconds). After the PREDETERMINED SLEEP TIME, the sensor node 720 renters the DEVICE STARTUP and the process is repeated.

If the battery voltage is above the CRITICAL BATTERY THRESHOLD, then the sensor nodes 720 measures the gas flow rate and then measures the 3-Axis of the accelerometer.

If the battery determination is false (e.g. SUFFICIENT BATTERY POWER is not present), then the system initializes the BARCODE reader, measures the revolutions per minute (“RPM”) from the Variable Frequency Drive (“VFD”) such as via a rotary sensor, and then measures the laser range distance. Subsequently, a sensor 730 measures the 3-Axis of the accelerometer.

In most normal operation, if there is SUFFICIENT BATTERY POWER, the sensor nodes 720 will operate such that they are measuring and recording various sensor readings. In other words, if the battery voltage is within the normal operating region, then the system will measure values from the units drive system, measure the proximity in front of the device, and read out any changes to the vehicle's position (such as represented by reading fixed barcodes on the rail as the vehicle drives along).

After measuring the 3-Axis of the accelerometer, regardless of the battery determination outcome, the sensors 730 measures and then stores the environmental pressure, humidity and temperature.

After storing the measurements, the system determines whether the battery voltage is below LOW-BATTERY THRESHOLD.

If the battery voltage is below the LOW-BATTERY THRESHOLD, then the sensor node 720 may alert such as by flashing a red light on a status LED.

If the battery voltage is not below the LOW-BATTERY THRESHOLD, then the sensor node 720 may alert such as by turning the status LED blue.

After issuing an alert, the system determines whether the gateway device 710 is available such as via Bluetooth.

If the gateway device 710 is reachable, then the sensor node 720 transfers “n” number of sensor 730 measurements to the gateway device 710 to be sent to server 30.

Then, the system determines whether any of the sensor 730 measurements exceeded FULL SAMPLE SPEED THRESHOLD. This threshold may differ depending on the type of measurement but generally refers to the maximum rate that data can be collected from all sensors 730.

If the gateway device 710 is not reachable, then the system proceeds to the FULL SAMPLE SPEED THRESHOLD determination.

If any of the sensor 730 measurements exceeded the FULL SAMPLE SPEED THRESHOLD, then the system reverts to DEVICE STARTUP and the process repeats.

If none of the sensor 730 measurements exceeded the FULL SAMPLE SPEED THRESHOLD, then the system enters DEEP-SLEEP MODE as discussed above.

With any of the measurements and sensors discussed regarding this process, the order, measurement types, and frequency may differ depending on system requirements.

### Embodiment 3

In another embodiment, an asset tracking system can be utilized in a safety application, such as to improve dock safety. Exemplary applications of this embodiment are shown in FIG. 9.

As shown in FIG. 8, for example, the system 900 may include access points 920 and server 930 as with the embodiments discussed above.

Additionally, a dock safety mobile unit 910, the same or similar to the mobile unit 10 discussed above, could be a human wearable of the Dock Safety system 900. The dock safety mobile unit 910 could also capitalize on the above discussed UWB and BT capabilities.

The system 900 also includes a dock safety PIV unit 950 which may be attached to a power industrial device (PIV). The dock safety PIV unit 950 may be similar to the mobile unit 910, but also may include additional functionality. For example, the dock safety PIV unit 950 may be hardwired into the PIV, and therefore not require battery/charging features, and may also include functionality to alert the driver of an impending contact event and/or collision.

As shown in example A (left), when the mobile unit 910 wearer and the dock safety PIV unit 950 are outside of a threshold distance, the system is not in alert. Conversely, as shown in example B (right), when the mobile unit 910 wearer and the dock safety PIV unit 950 are within a threshold distance, the system is in alert.

These alerts could be visual, audial, and/or haptic and could include varying levels of alert. For example, when the dock safety PIV unit 950 approaches someone with a dock safety mobile unit 910 or another dock safety PIV unit 950, the dock safety PIV unit 950 could illuminate a LED or an attached stack light. Similarly, as the dock safety PIV 950 unit gets closer, the LED or stack light color could change until it reaches a higher alert level color such as solid RED. If the dock safety PIV unit 950 gets within a settable distance to another dock safety PIV unit or dock safety PIV mobile unit the LED or stack light color changes, the LED or stack light could flash RED.

Additionally, dock safety PIV unit 950 could include external device control functionality to control the equipment such as emergency cut-off functionality in the case of mobile equipment. For example, in the case of a fork-lift in a contact or near-contact event, the system may be capable of equipment shutoff to prevent a collision or other damage.

Similarly, when the dock safety mobile unit 910 approaches or is approached by a PIV equipped with a dock safety PIV unit 950, the dock safety mobile unit may include functionality to both log the event and alert the carrier of the dock safety mobile unit 910. For example, the dock safety mobile unit 910 could include an LED, which will change colors and start vibrating based on range. As the dock safety PIV unit enabled PIV such as a fork life gets closer to the person wearing or holding the dock safety mobile unit, the LED color will change colors until it reaches RED and the vibration bursts will increase in frequency until it is non-stop.

As with the embodiments discussed above, the mobile unit 910 wearer and the dock safety PIV unit 950 may be configured to only upload the stored data to server 930 when they are within reach of an access point 920. Accordingly, the dock safety system may be used in environments with limited or restricted wireless communications. And, like other of the present systems, the dock safety PIV unit 950 and dock safety mobile unit 910 may communicate to the server via existing infrastructure using network access points such as WIFI gateways.

Other wireless dock safety systems rely on signal strength and audible alerts for proximity. Those systems are not able to differentiate their responses based on actual distance but rely on received signal strength. Those systems depend on adequately charged batteries in their wireless transmitters. The dock safety PIV unit 950 or dock safety mobile units 910 do not necessarily rely on received signal strength for function.

Another adaptable feature is that the dock safety PIV unit 910 is that it also may be installed in a fixed manner at traffic intersections or blind corners to act as fixed warnings as dock safety PIV unit 950 enabled PIVs or dock safety mobile unit 910 wearing people approach those locations.

Additionally, audible alerts often go unnoticed by wearers due to high level of noise in productive environments, so the varying levels and options of alerts provide additional levels of protection.

A dock safety system may also pull together the functions of the dock safety PIV unit 950 and dock safety mobile unit 910 by communicating interaction data back to a dock safety server for the use of safety records/visualizations and for dock traffic flow optimization improvements.

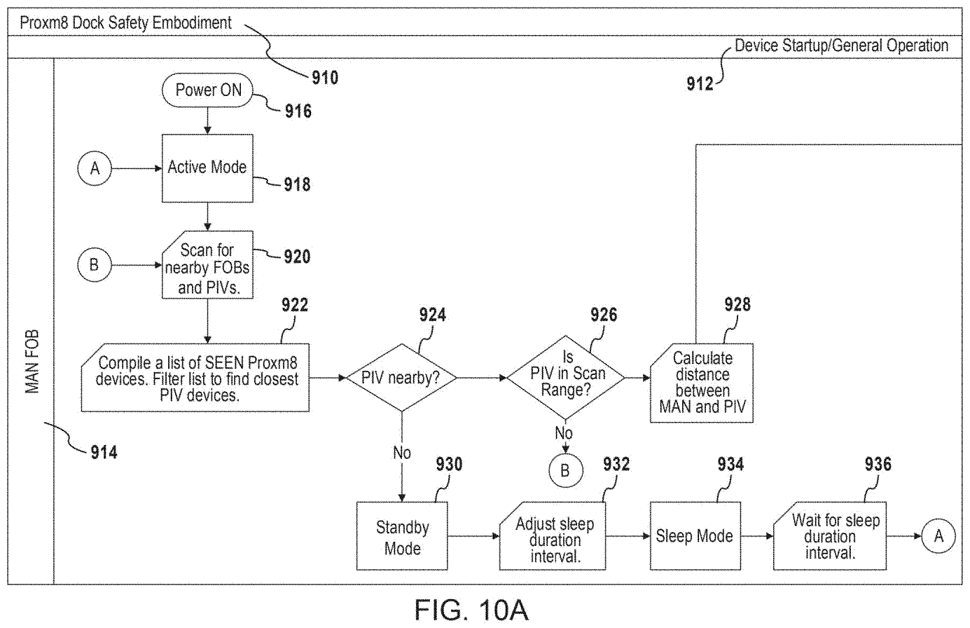

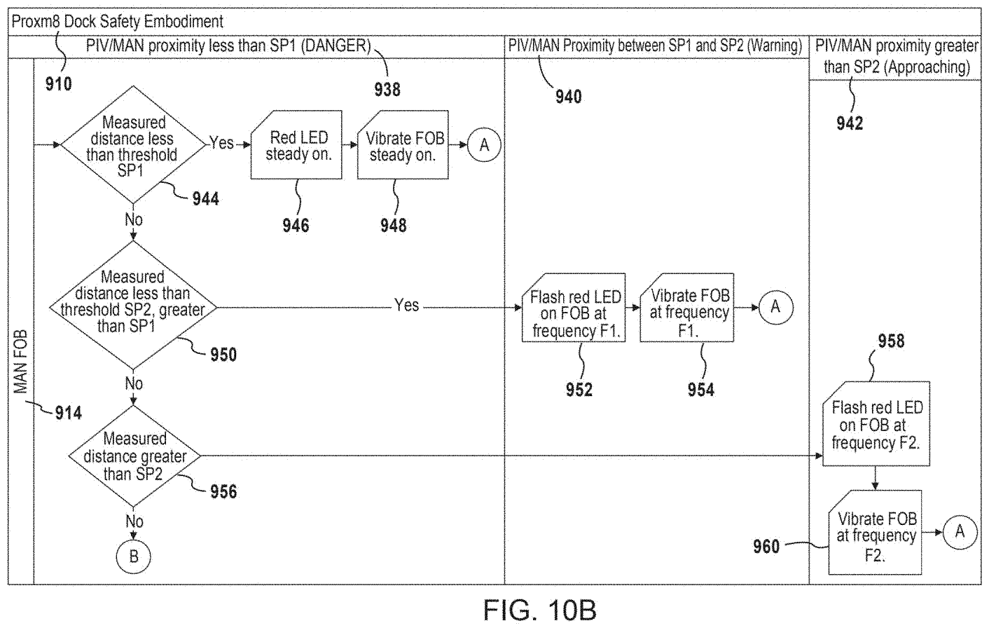

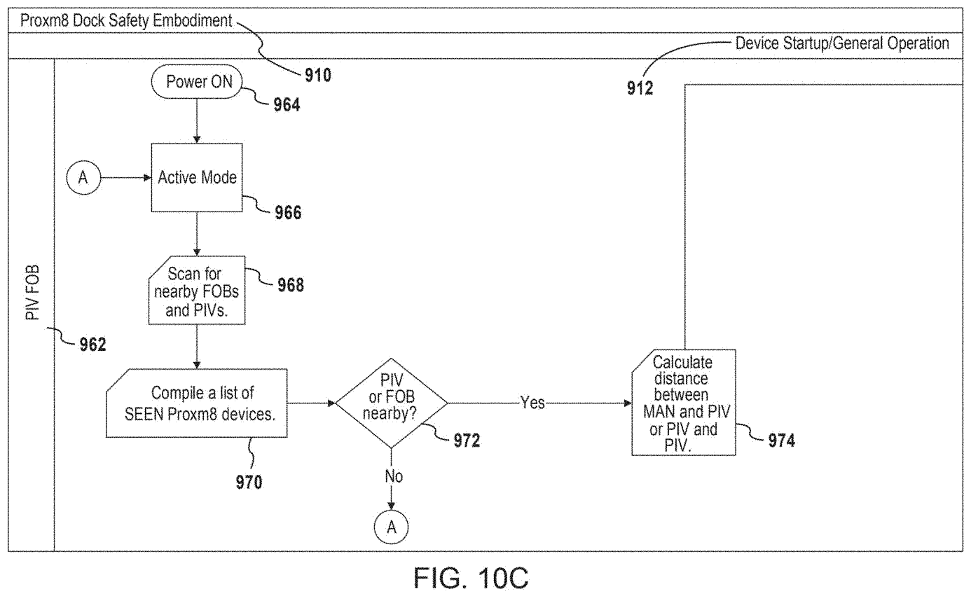

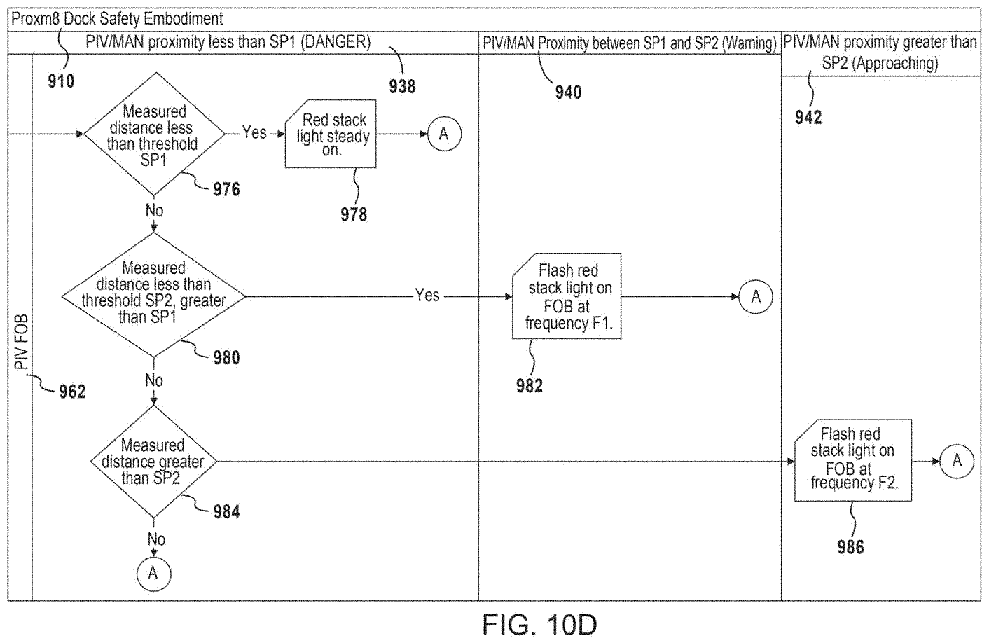

FIGS. 10A-C shows a flowchart of exemplary detection and analysis performed by the system in a dock safety application at different events points within the process.

FIG. 10B, which is the top half of FIG. 10A, shows the processes for the dock safety mobile unit 910.

As shown in FIG. 10B, when the power is turned on, the dock safety mobile unit 910 enters ACTIVE MODE and then scans for any nearby dock safety mobile units 910 or dock safety PIV units 950.

After scanning, the dock safety mobile unit 910 compiles a list of SEEN mobile units 910 and dock safety PIV units 950 that were obtained running the scan and filters the results to find the closest dock safety PIV units 950.

The dock safety mobile unit 910 then determines whether there is a dock safety PIV units 950 nearby.

If there is no dock safety PIV units 950 nearby, then the dock safety mobile unit 910 enters STANDBY MODE, adjusts the SLEEP DURATION INTERVAL, enters SLEEP MODE, and then waits for the SLEEP DURATION INTERVAL before re-entering ACTIVE MODE and repeating the process.

If there is a dock safety PIV unit[s] 950 nearby, the dock safety mobile unit 910 determines whether the dock safety PIV unit 50 is within SCAN RANGE.

If the dock safety PIV unit 950 is not within SCAN RANGE, then the dock safety mobile unit 910 performs the scan for any nearby dock safety mobile units 910 or dock safety PIV units 950 and repeats the process.

If the dock safety PIV unit 950 is within SCAN RANGE, then the dock safety mobile unit 910 calculates the distance between the dock safety PIV unit 950 and the dock safety mobile unit 910. As discussed above and described in detail below, the alerts may have levels changing based on certain distances and system requirements.

For example, if the MEASURED DISTANCE is less than THRESHOLD SP1, then the dock safety mobile unit 910 issues an alert such as having the status light maintain a steady red and then vibrate at a steady level for a certain amount of time. Then, the dock safety mobile unit 910 reenters ACTIVE MODE and the process is repeated.

If the MEASURED DISTANCE is greater than THRESHOLD SP1 but less than THRESHOLD SP2, then the dock safety mobile unit 910 issues an alert such as having the status light maintain a flash red at frequency F1 and then vibrate at a frequency F1 for a certain amount of time. Then, the dock safety mobile unit 910 reenters ACTIVE MODE and the process is repeated.

If the MEASURED DISTANCE is greater than THRESHOLD SP2, then the dock safety mobile unit 910 issues an alert such as having the status light maintain a flash red at frequency F2 and then vibrate at a frequency F2 for a certain amount of time. Then, the dock safety mobile unit 910 reenters ACTIVE MODE and the process is repeated.

If the MEASURED DISTANCE is not greater than SP2, then the dock safety mobile unit 910 performs the scan for any nearby dock safety mobile units 910 or dock safety PIV units 950 and repeats the process.

FIG. 10C, which is the top half of FIG. 10A, shows the processes for the dock safety PIV unit 950.

As shown in FIG. 10C, when the power is turned on, the dock safety PIV unit 950 enters ACTIVE MODE and then scans for any nearby dock safety mobile units 10 or dock safety PIV units 950.

After scanning, the dock safety PIV unit 950 compiles a list of SEEN mobile units 910 and dock safety PIV units 950 that were obtained running the scan.

The dock safety PIV unit 950 then determines whether there is a dock safety PIV unit 950 or dock safety mobile unit 910 nearby.

If there is no dock safety PIV units 950 or dock safety mobile unit 910 nearby, then the dock safety PIV unit 950 reenters the ACTIVE MODE and repeats the process.

If there is a dock safety PIV unit 950 or dock safety mobile unit 910 nearby, then the dock safety PIV unit 910 calculates the distance between the dock safety PIV unit 950 and the nearby device. As discussed above and described in detail below, the alerts may have levels changing based on certain distances and system requirements.

For example, if the MEASURED DISTANCE is less than THRESHOLD SP1, then the dock safety PIV unit 950 issues an alert such as having the stack light maintain a steady red. Then, the dock safety PIV unit 950 reenters the ACTIVE MODE and the process is repeated.

If the MEASURED DISTANCE is greater than THRESHOLD SP1 but less than THRESHOLD SP2, then the dock safety PIV unit 950 issues an alert such as having the status light on the dock safety mobile unit 910 maintain a flashing red at frequency F1 for a certain amount of time. Then, the dock safety PIV unit 950 reenters the ACTIVE MODE and the process is repeated.

If the MEASURED DISTANCE is greater than THRESHOLD SP2, then the dock safety PIV unit 950 issues an alert such as having the status light on the dock safety mobile unit 910 maintain a flashing red at frequency F2 for a certain amount of time. Then, the dock safety PIV unit 950 reenters ACTIVE MODE and the process is repeated.

If the MEASURED DISTANCE is not greater than SP2, then the dock safety PIV unit 950 reenters the ACTIVE MODE and the process is repeated.

### Embodiment 4

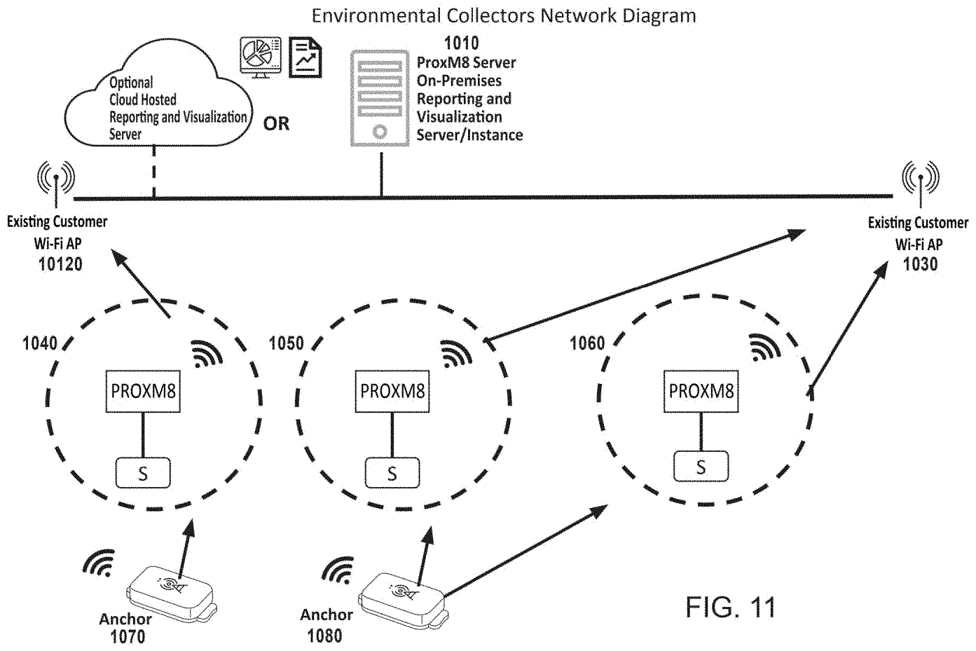

In another embodiment, an asset tracking system can be utilized in an environmental collection application. An example of such a system is shown in FIG. 11.

Whereas, other systems only collect and forward sensor data of sensors that are physically connected to the device, the asset tracking environmental collector shown in FIG. 11 and described herein is designed to collect data from attached or wirelessly connected sensory devices (e.g., mobile units 1110) and forward the data to an asset tracking server (e.g. server 1130). Additionally, in certain embodiments, the asset tracking environmental collector can also act as an anchor 1140 allowing it to aid in the localization of other mobile units 1110.

In some embodiments, the asset tracking environmental collector (e.g. mobile units 1110 and/or anchors 1140) could include other laser sensors, temperature measurement devices, humidity measurement devices, accelerometer, pressure and flow meters, and battery voltage monitoring.

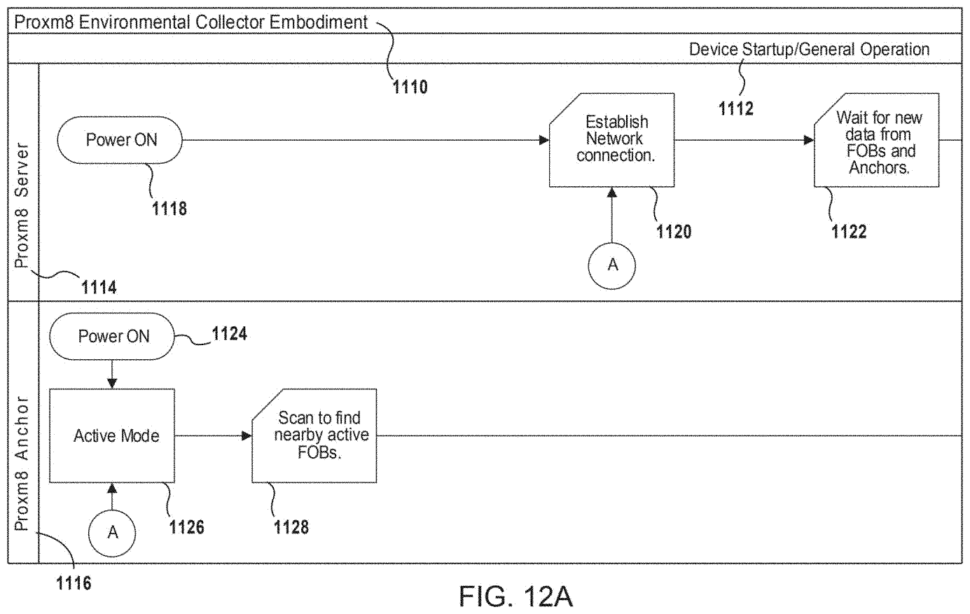

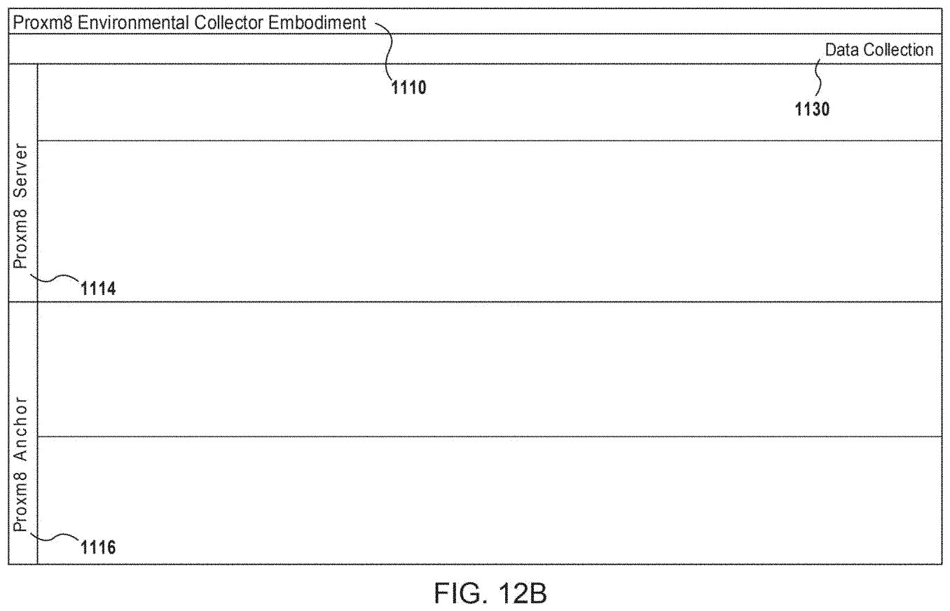

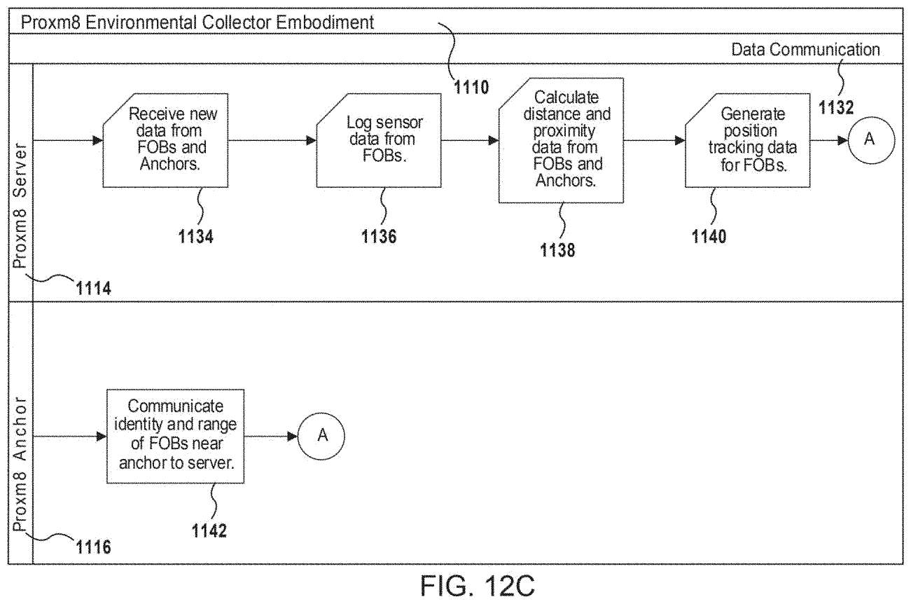

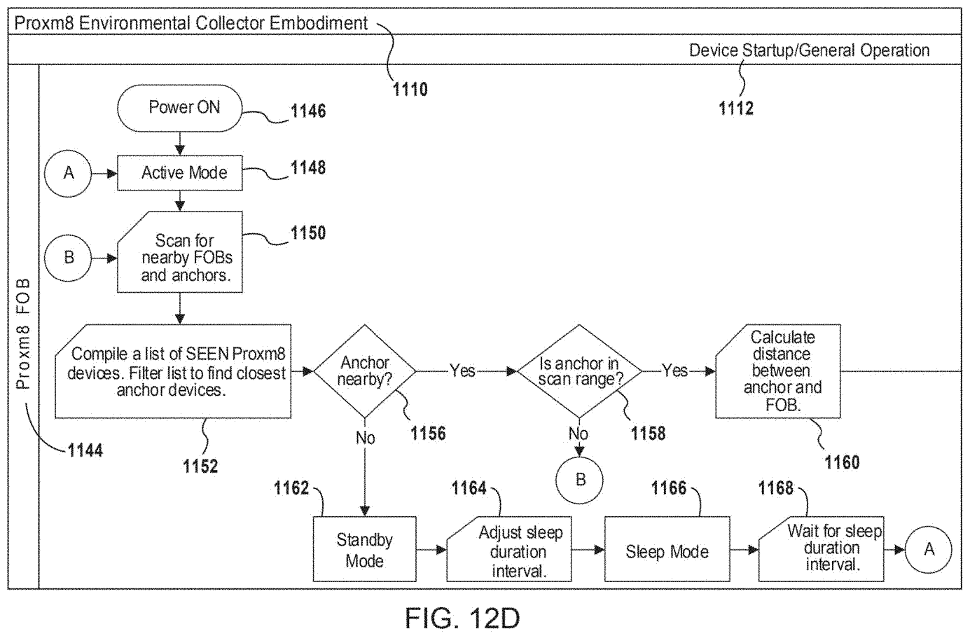

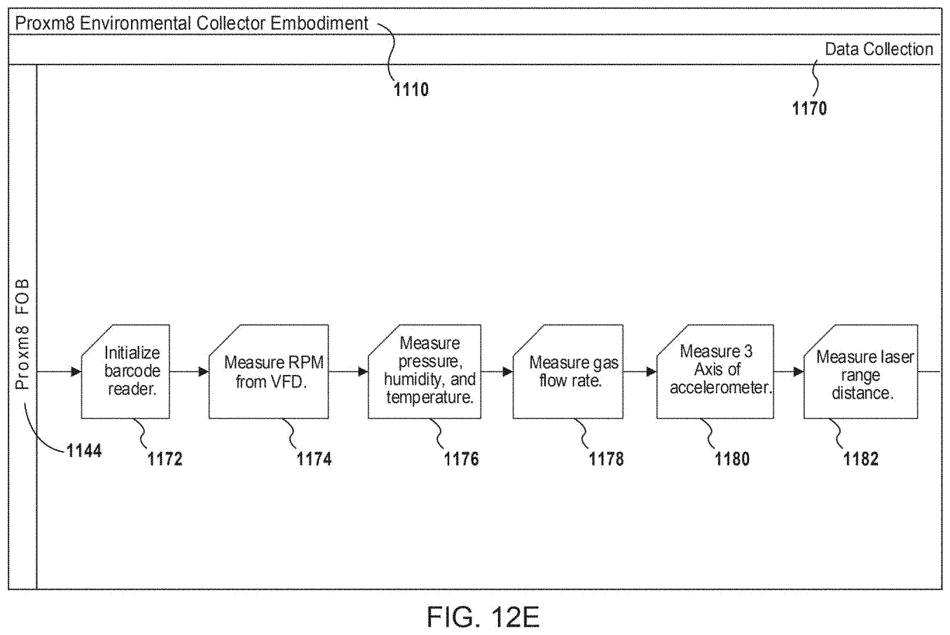

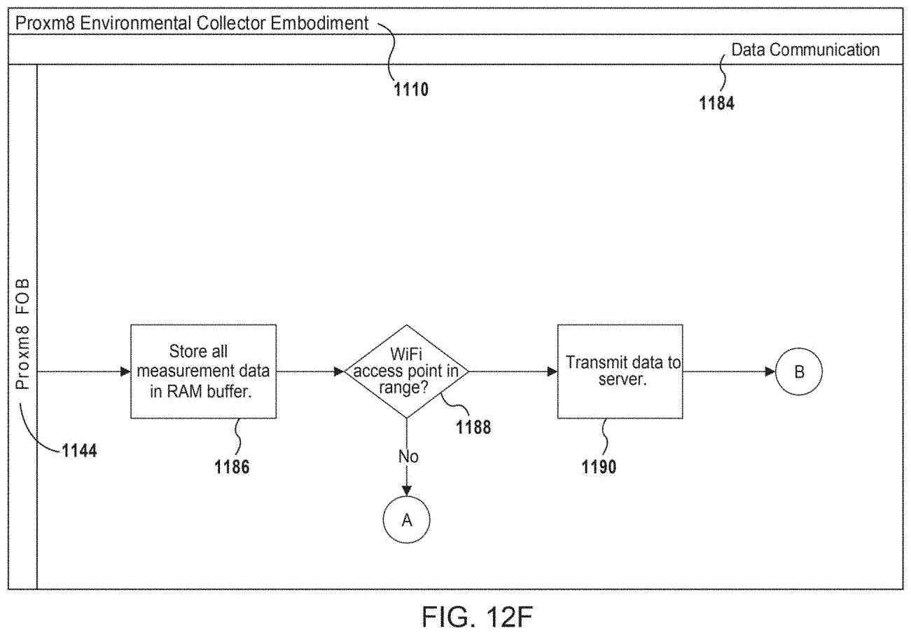

FIG. 12, for example, shows a flowchart of environmental collector application performed by the server 1130, anchors 1140, and mobile units 1110 at different events points within the process.

As shown in the top row of FIG. 12, at start up, the server 1130 powers on and then establishes a network connection. Once the network connection is established, the server 1130 waits for new information from the mobile units 1110 and anchors 1140.

After receiving data from the mobile units 1110 and/or anchors 1140, the server logs the sensor data from the mobile units 1110. The server 1130 then calculates the distance and proximity data from the mobile units 1110 and anchors 1140.

Next, the server 1130 generates position tracking data for the mobile units 1110 and then reverts to the establish network connection step and the process repeats.

As shown in the middle row of FIG. 12, at start up, the anchors 1140 enter ACTIVE MODE and then scans to find nearby active mobile units 1110.

Subsequently, the anchors 1140 communicate the identify and range of mobile units 1110 nearby anchor 1140 to the server 1130. Then, the anchor 1140 reenters ACTIVE MODE and the process repeats.

As shown in the bottom row of FIG. 12, at start up, the mobile unit 1110 enters ACTIVE MODE and proceeds to scan for nearby active mobile units 1110 and anchors 1140.

After scanning, the mobile unit 1110 compiles a list of SEEN mobile units 1110 and anchors 1140 that were obtained running the scan and filters the results to find the closest anchor 1140.

The mobile unit 1110 then determines whether there is an anchor 1140 nearby.

If there is no anchor 1140 nearby, then the mobile unit 1110 enters STANDBY MODE, adjusts the SLEEP DURATION INTERVAL, enters SLEEP MODE, and then waits for the SLEEP DURATION INTERVAL before re-entering ACTIVE MODE and repeating the process.

If there is an anchor 1140 nearby, the mobile unit 1110 determines whether the anchor 1140 is within SCAN RANGE.

If the anchor 1140 is not within SCAN RANGE, then the mobile unit 1110 performs the scan for any nearby dock safety mobile units 1110 or anchors 1140 and repeats the process.

If the anchor 1140 is within SCAN RANGE, then the mobile unit 1110 calculates the distance between the mobile unit 1110 and the anchor 1140.

Then, the mobile unit 1110 initializes the barcode reader and measures, for example, RPM from VFD, pressure, humidity, temperature, gas flow rate, 3-Axis of accelerometer, and laser range distance which are all then stored in a RAM buffer.

Then, mobile unit 1110 determines whether an access point 1120 is in range.

If an access point 1120 is not in range, then mobile unit 1110 reenters the ACTIVE MODE and the process repeats.

If an access point 1120 is in range, then the mobile unit 1110 transmits the data to server 1130 via access point 1120 and then reverts to the scan for nearby active mobile units 1110 and anchors 1140 step and the process repeats.

### Embodiment 5

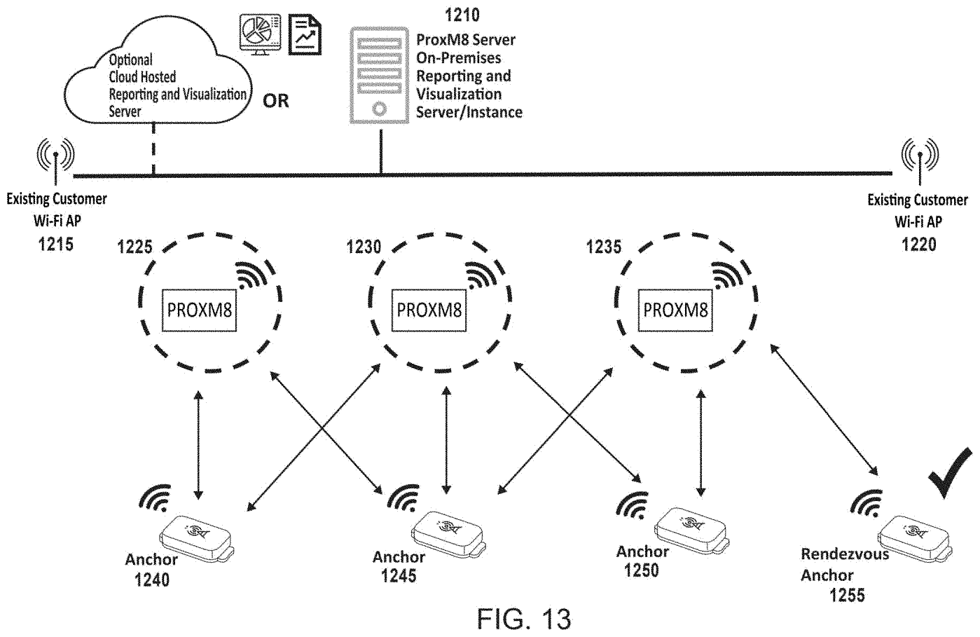

In another embodiment, as shown in FIG. 13, an asset tracking system can be utilized in an emergency preparedness and reaction tool application.

Natural disaster, fire hazards, and workplace violence (e.g., active shooter situation) are less frequent than other safety hazards that facilities face. Despite best efforts by facility safety teams to provided adequate training to personnel and visitors, there is nearly always a great deal of confusion and slow response to such event alarms. Generally, facility safety teams establish visitor training processes, post exit maps throughout the facility, and run periodic drills. An asset tracking emergency preparedness and reaction tool system use mobile units 1310, anchors 1340, access points 1320, and asset tracking servers 1330 as components to reduce confusion and promote a rapid response solution to natural disaster, fire hazard event, workplace violence event.

When someone initiates an alarm, such as pulling a fire alarm or starting a tornado alarm or other, the alarm triggers the asset tracking server 1330 to act. Based on the alarm type, the asset tracking server 1330 application will execute a pre-defined response strategy. Response strategies could include mobile unit 1310 based notification of event, location of rendezvous points, or evacuation routes. The mobile units 1310 can be configured to vibrate to catch wearer attention and can illuminate an LED as discussed with other embodiments above.

Moreover, the system could be configured to give alerts to the wearer of the mobile unit 1310 to avoid dangerous locations and/or direct them to safe locations. For example, the color of the LED on the mobile unit 1310 could indicate direction that the wearer is expected to go to reach a safe location. The direction is determined by the asset tracking server 1330 application and the location of the mobile unit 1310 wearer as well as location and environment data (such as discussed regarding the Environmental Collectors embodiment above) that may be obtained from other mobile units 1310 and/or anchors 1340. For example, when a mobile unit 1310 wearer goes the wrong direction, the LED can flash a color and when the wearer is moving in the correct direction, the color could remain solid. Once the wearer arrives at the rendezvous/safe point, the asset tracking server 1330 application logs once the wearer is safe.

Facility safety teams will also be able to view real time the location of each mobile unit 1310 wearer during the event and identify unaccounted-for wearers. Plus, facility safety teams will be able to review/replay response events to optimize evacuation routes and rendezvous points.

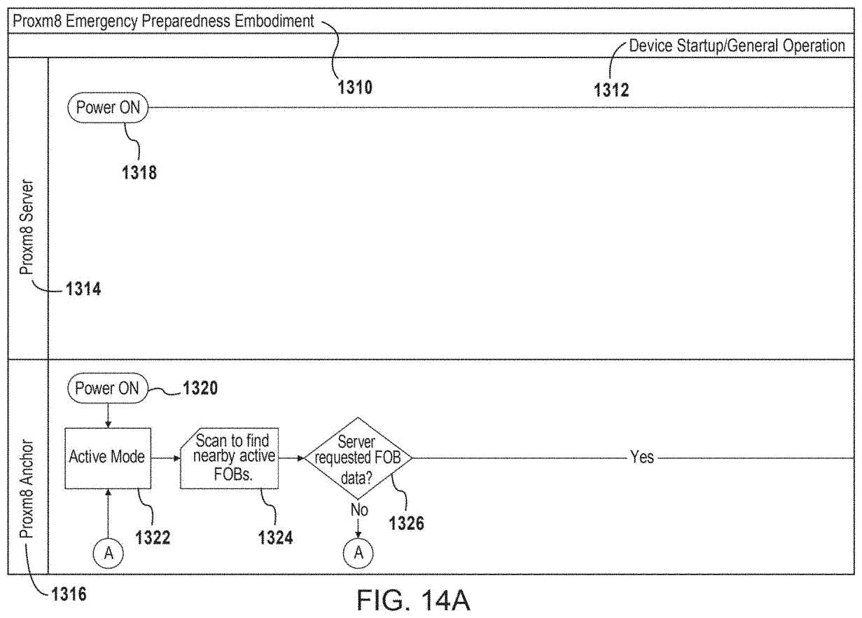

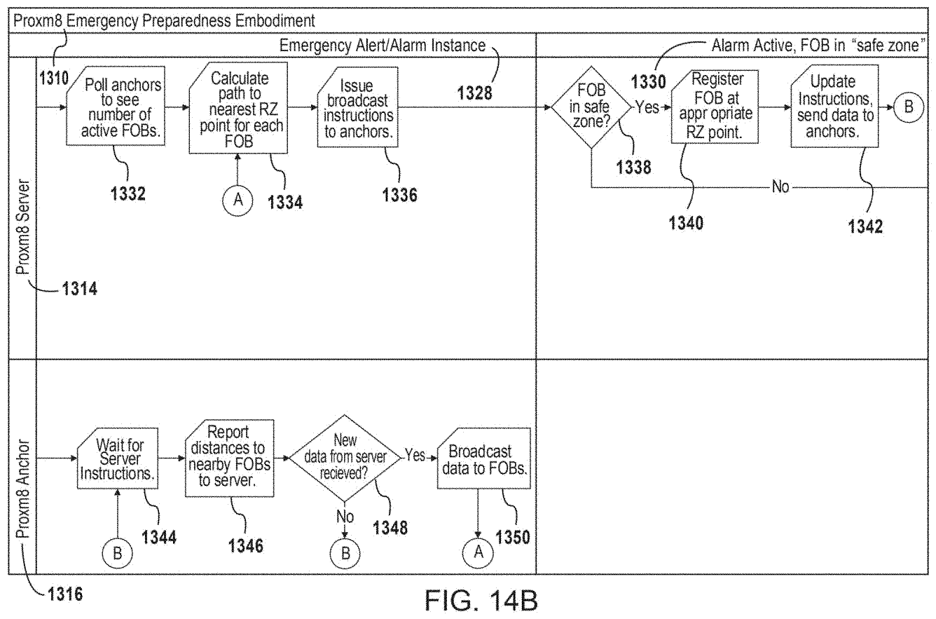

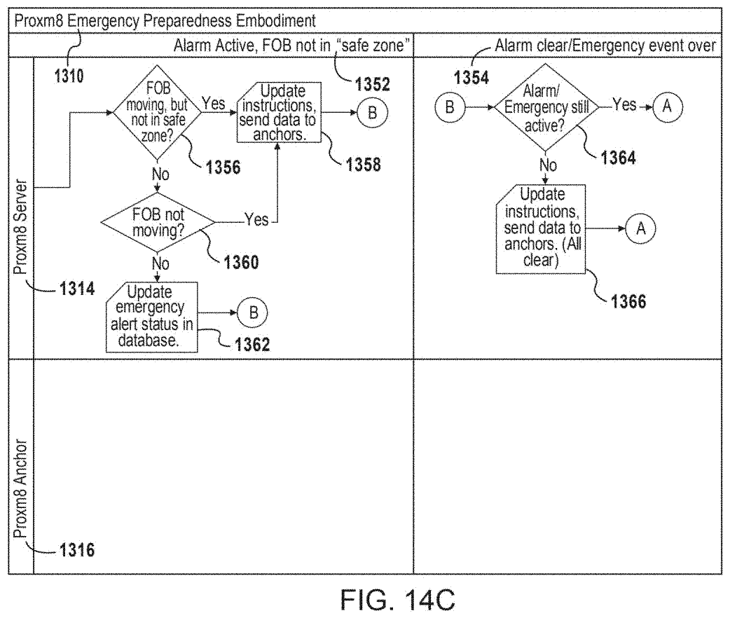

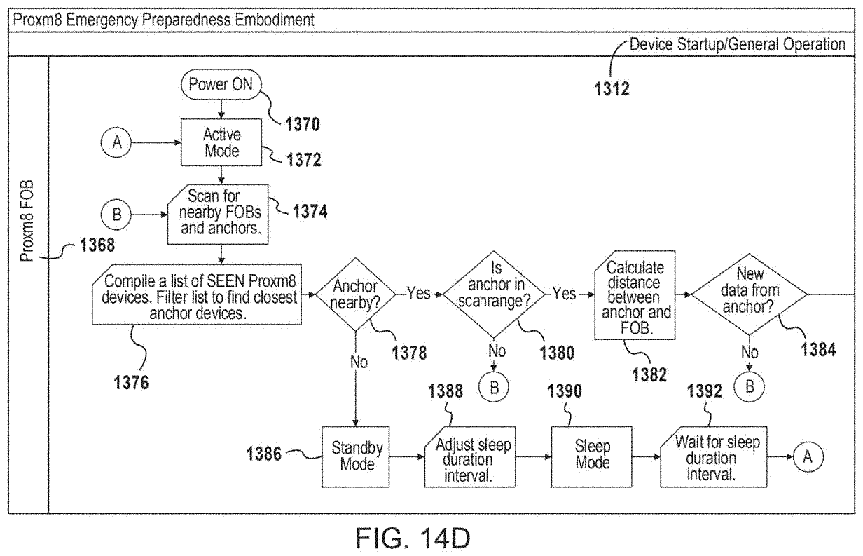

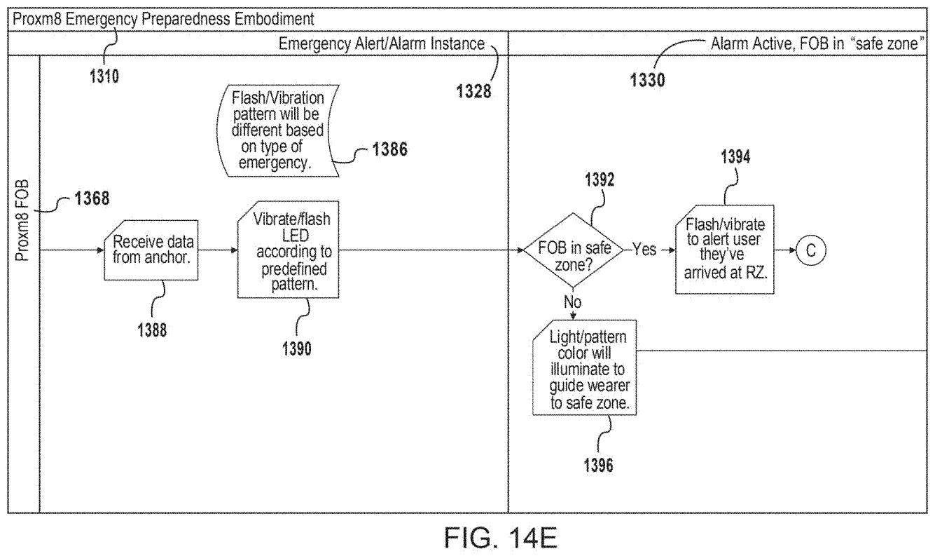

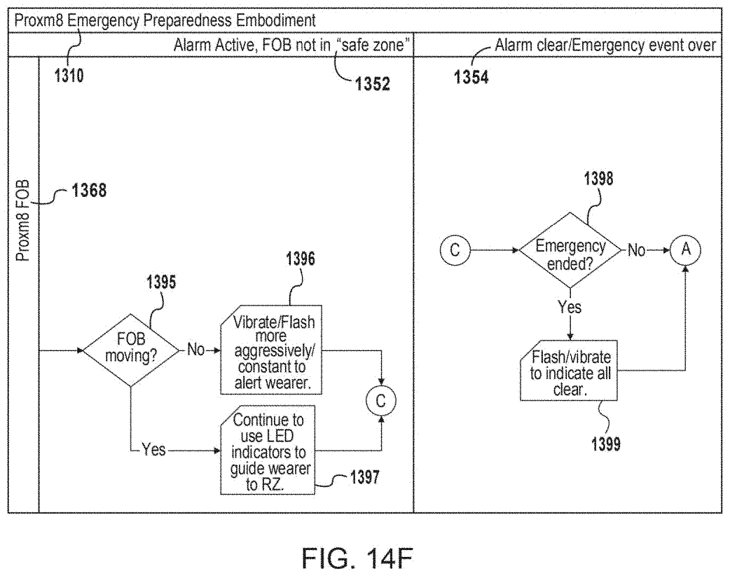

FIG. 14 shows a flowchart of exemplary emergency preparedness application performed by the server 1330, anchors 1340, and mobile units 1310 at different events points within the process.

As show in the top of FIG. 14, after power on, in an emergency alert/alarm instance, the server 1330 polls the anchors 1340 to see the number of active mobile units 1310.

Then, the server 1330 calculates a path to the nearest rendezvous and/or safety point for each active mobile unit 1310. Subsequently, the server 1330 issues broadcast instructions for the mobile units 1310 to the anchors 1340.

Then, the server 1330 determines whether the mobile unit 1310 is in the safe zone.

If the mobile unit 1310 is in the safe zone, then the server 1330 registers the mobile unit 1310 as located at the appropriate rendezvous point. Then, the server 1330 updates the instructions, broadcasts the updated instructions to the anchors 1340, and then enters the alarm clear/emergency event over determination stage.

If the mobile unit 1310 is not in the safe zone, then the server 1330 determines whether the mobile unit 1310 is moving. If the mobile unit is moving, then the server 1330 updates the instructions, broadcasts the updated instructions to the anchors 1340, and then enters the alarm clear/emergency event over determination stage.

If the mobile unit 1310 is not moving, then the server 1330 updates the EMERGENCY ALARM STATUS in the database and then enters the alarm clear/emergency event over determination stage.

At the alarm clear/emergency event over determination stage, the server 1330 determines whether the alarm/emergency is still active. If it is, then server 1330 reverts to the calculate a path to the nearest rendezvous and/or safety point for each active mobile unit 1310 step. If the alarm/emergency is not active, then the server 1330 updates the instructions, broadcasts the updated instructions to the anchors 1340, and then reverts to the calculate a path to the nearest rendezvous and/or safety point for each active mobile unit 1310 step.

As show in the middle row of FIG. 14, after power on, the anchor 1340 enters ACTIVE MODE and proceeds to scan for nearby active mobile units 1310.

Then, the anchor 1340 determines whether the server 1330 requested mobile unit 1310 data.

If the server 1330 did not request mobile unit 1310 data, then the anchor 1340 reverts to the ACTIVE MODE and the process repeats.

If the server 1330 did request mobile unit 1310 data, then the anchor 1340 waits for instructions from server 1330.

After receiving instructions from server 1330, the anchor 1340 reports distances of nearby mobile units 1310 to server 1330.

Then, if there is no new data received from server 1330, then anchor 1340 reverts to the wait for instructions from server 1330 step.

If there are new instructions from server 1330, then the anchors 1340 broadcasts the data to mobile units 1310 and reverts to the ACTIVE MODE and the process repeats.

As show in the bottom row of FIG. 14, after power on, the mobile unit 1310 enters the ACTIVE MODE and proceeds to scan for nearby active mobile units 1310 and anchors 1340.

After scanning, the mobile unit 1310 compiles a list of SEEN mobile units 1310 and anchors 1340 that were obtained running the scan and filters the results to find the closest anchor 1340.

The mobile unit 1310 then determines whether there is an anchor 1340 nearby.

If there is no anchor 1340 nearby, then the mobile unit 1310 enters STANDBY MODE, adjusts the SLEEP DURATION INTERVAL, enters SLEEP MODE, and then waits for the SLEEP DURATION INTERVAL before re-entering ACTIVE MODE and repeating the process.

If there is an anchor 1340 nearby, the mobile unit 1310 determines whether the anchor 1340 is within SCAN RANGE.

If the anchor 1340 is not within SCAN RANGE, then the mobile unit 1310 performs the scan for any nearby dock safety mobile units 1310 or anchors 1340 and repeats the process.

If the anchor 1340 is within SCAN RANGE, then the mobile unit 1310 calculates the distance between the mobile unit 1310 and the anchor 1340.

Then, the mobile unit 1310 determines whether there is new data from the anchor 1340.

If there is no new data, then the mobile unit 1310 performs the scan for any nearby dock safety mobile units 1310 or anchors 1340 and repeats the process.

If there is new data, then the mobile unit 1310 receives the data from the anchor 1340 and issues an alert consistent with the instruction, such as vibrating or flashing an LED according to a predefined pattern.

Then the mobile unit 1310 determines whether the mobile unit 1310 is in a safe zone.

If the mobile unit 1310 is in a safe zone, then it will alert indicating that the mobile unit 1310 is in the rendezvous zone and proceed to the emergency ended determination step.

If the mobile unit 1310 is not in a safe zone, then the mobile unit 1310 will issue an alert to guide the wearer to the safe zone.

Then, the mobile unit 1310 will determine whether the mobile unit 1310 is moving or stationary.

If the mobile unit 1310 is moving, then the mobile unit 1310 will continue to use the alerts (e.g., LED) to guide the user to the safe zone and then proceed to the emergency ended determination step.

If the mobile unit 1310 is not moving (e.g., stationary or mostly stationary), then the mobile unit 1310 will alert more aggressively (e.g., more vibrations), and then proceed to the emergency ended determination step.

At the emergency ended step, the mobile unit 1310 determines whether the emergency has ended. If the emergency has ended, then the mobile unit 1310 alerts to indicate all clear and then proceeds to ACTIVE MODE and the process repeats.

If the emergency has not ended, then the mobile unit 1310 proceeds to ACTIVE MODE and the process repeats.

### Embodiment 6

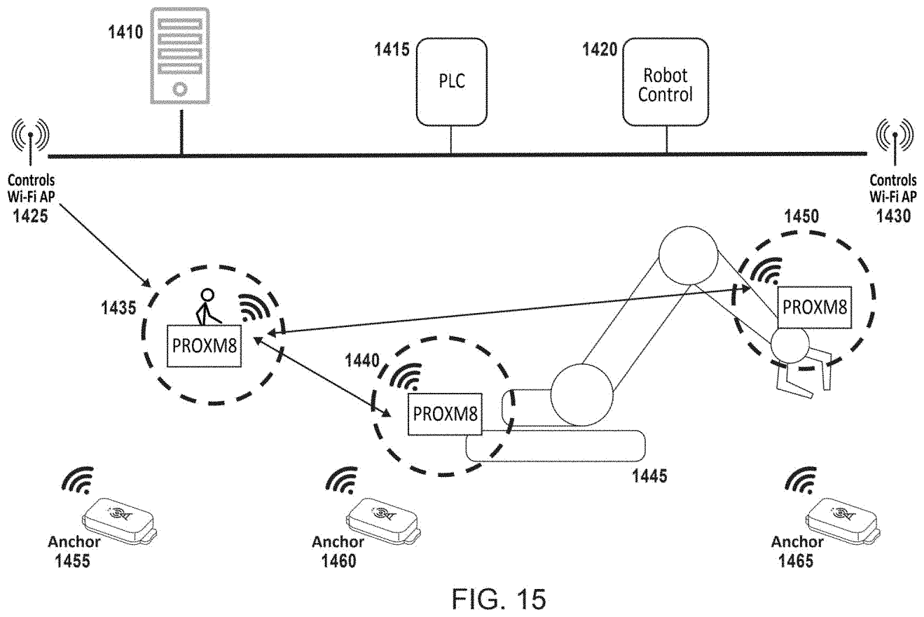

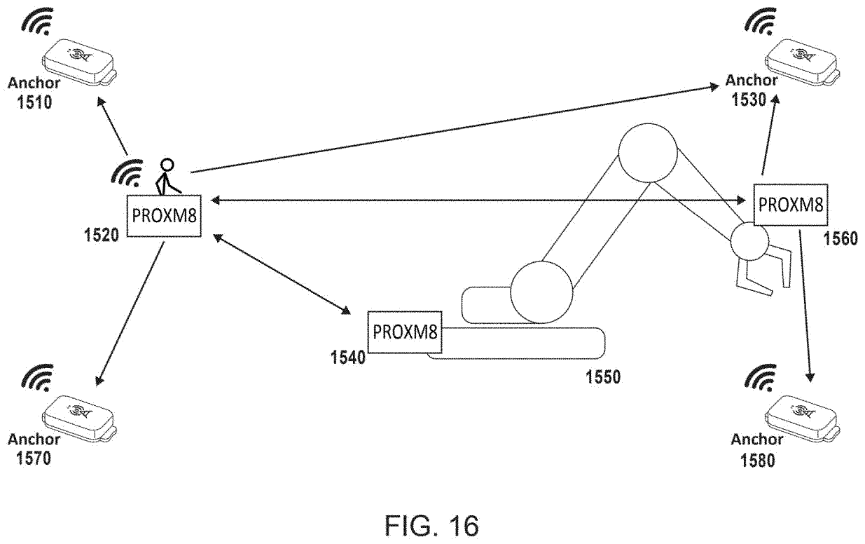

In another embodiment, as shown for example in FIGS. 15-16, an asset tracking system can be utilized in a robot proximity monitoring application.

An asset tracking robot proximity monitoring unit 1510 (e.g., mobile unit 10) is designed, for example, to be a subcomponent to a robot cell safety system. In conjunction with fixed mounted anchors 1540 and robot mounted anchors 1545, the robot proximity monitoring unit 1510 is configured to provide location information of an authorized person who enters a robot cell to a robot controller.

The asset tracking system allows for variable safety zones to be established with the robot. In this way, the robot speed and operation can be modulated to accommodate the presence of the wearer without stopping. Robot speed can be reduced as the distance between the robot's end of arm tool and the wearer is reduced. The robot could be stopped if the distance becomes less than a preset threshold established by the required robot safety risk assessment. The robot speed reduction would also be based on established safety distance requirements. When the wearer is in the safety cell, light curtains, floor scanners, and other safety detection devices can be muted depending on wearer location. A robot safety risk assessment is required and proper ancillary safety devices such as light curtains, floor scanners, et cetera must be selected with features that allow for application appropriate level of spatial granularity. This is necessary to ensure that those ancillary safety devices function properly when a robot proximity monitoring unit wearer is in the cell and a NON-wearer tries to enter.

Currently, robot safety cells are limited to bulky safety zones which slow the robot prematurely or stop the robot all together when an authorized operator is in the area. The present asset tracking system allows for finer resolution of approach. For instance, a typical safety zone might be 2 m×2 m and positioned 1 m from the robot. When the operator steps into the edge of the zone farthest from the robot, the robot drops from nominal speed to some safe speed appropriate for 1 m of clearance. With this asset tracking system, the speed can be metered down based on where the operator is from 3 m to 1 m from the robot.

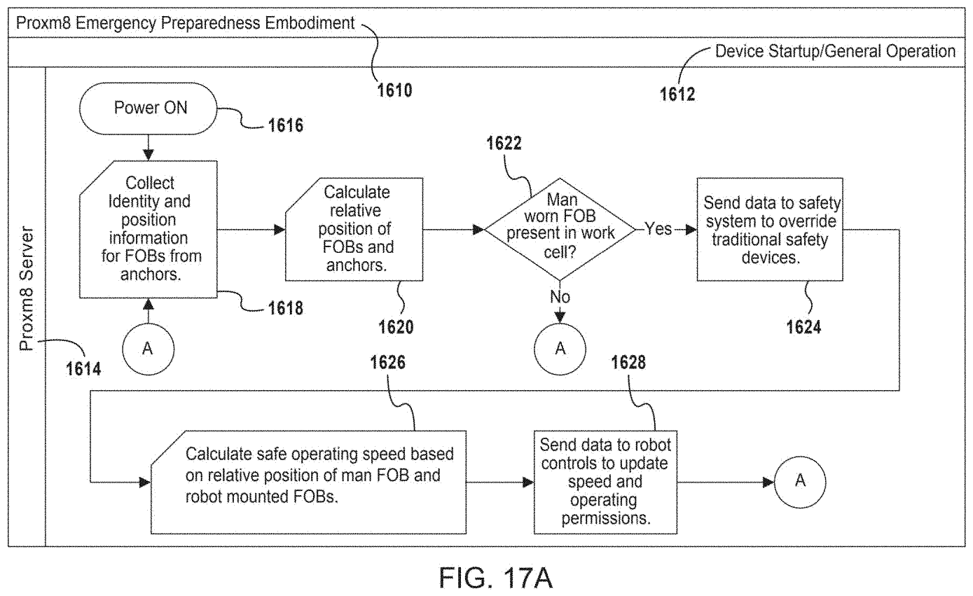

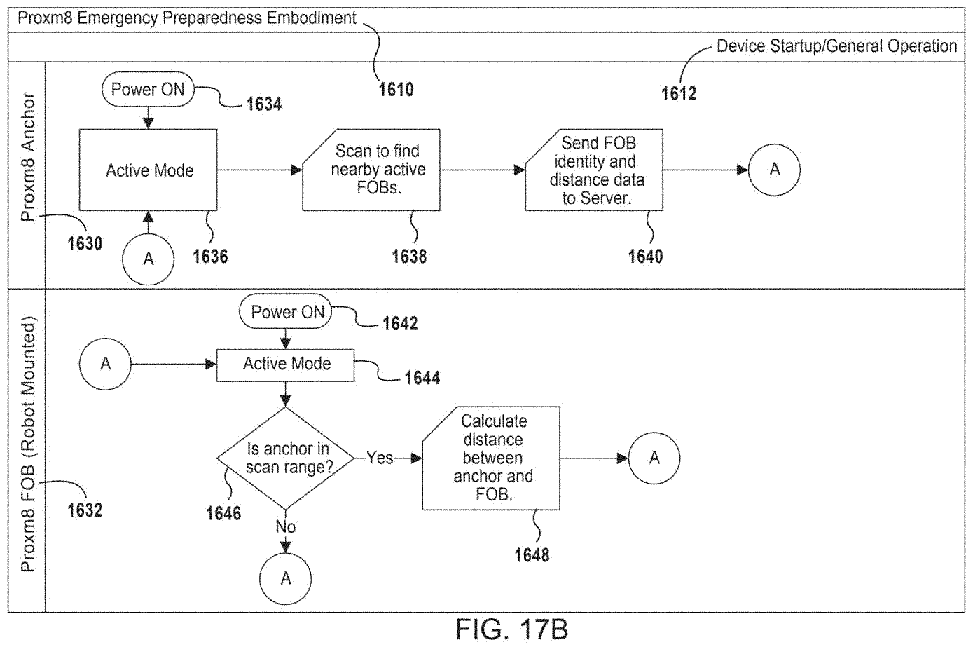

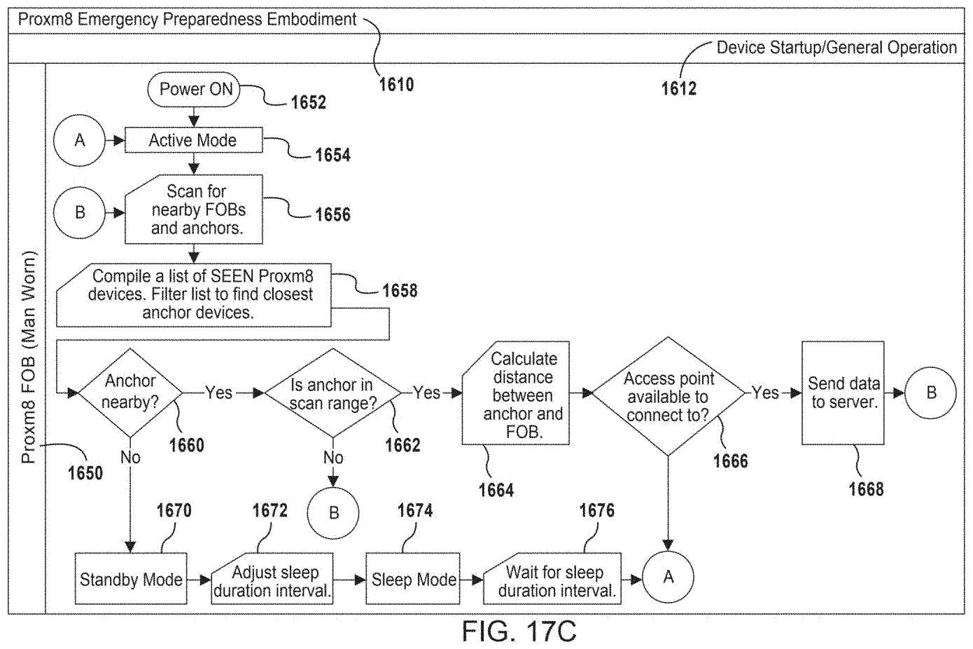

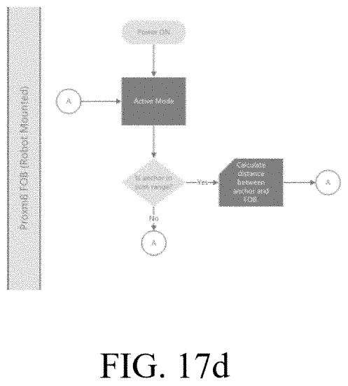

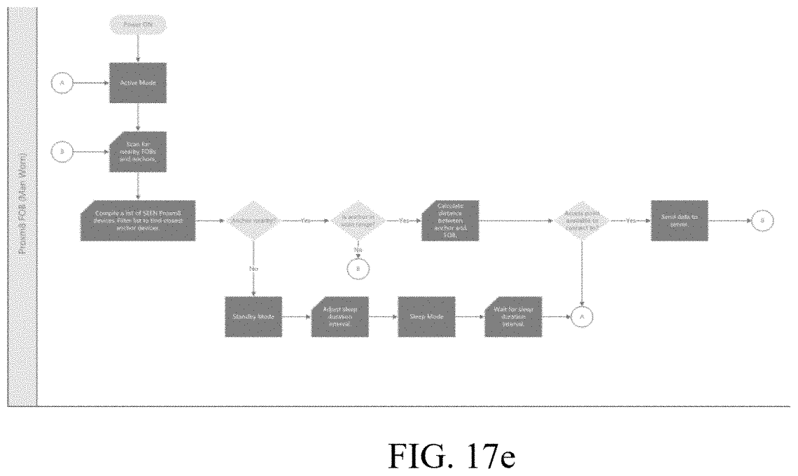

FIG. 17A shows a flowchart of exemplary robot safety application performed by the server 1530 (expanded in FIG. 17B), anchors 1540 (expanded in FIG. 17C), robot-mounted anchors 1545 (expanded in FIG. 17D), and mobile units 1510 (expanded in FIG. 17E) at different events points within the process.

As shown in FIG. 17B, at power on, server 1530 collects identity and position information for mobile units 1510, anchors 1540, and robot-mounted anchors 1545.

Then, server 1530 calculates a relative position of mobile units 1510, anchors 1540, and robot-mounted anchors 1545.

Next, server 1530 determines whether a person-worn mobile unit 1510 is present within a work cell.

If no person-worn mobile unit 1510 is present within the work cell (e.g., predetermined area around robot-mounted anchor 1545), then the server 1530 reverts to the power on step and the process repeats.

If a person-worn mobile unit 1510 is present within the work cell, then the server 1530 sends data to the safety system to override the current safety devices.

The server 1530 then calculates a safe operating speed based on the relative position of the man-worn mobile unit 1510 and the robot-mounted anchor 1545. Then, server 1530 sends this data to the robot controls to update robot speed and operating permissions.

Finally, the server 1530 reverts to the power on step and the process repeats.

As shown in FIG. 17C, at power on, the anchor 1540 enters ACTIVE MODE and then scans to find nearby active mobile units 1510 and robot mounted anchors 1545.

The anchor 1540 then sends the nearby active mobile units 1510 and robot mounted anchors 1545 identity and distance data to the server 1530 before re-entering the ACTIVE MODE and repeating the process.

As shown in FIG. 17D, at power on, the robot-mounted anchor 1545 enters the ACTIVE MODE and then determines whether an anchor 1540 is within SCAN RANGE.

If no anchor 1540 is within the SCAN RANGE, then the robot-mounted anchor 1545 reenters the ACTIVE MODE and the process repeats.

If an anchor 1540 is within the SCAN RANGE, then robot-mounted anchor 1545 calculates the distance between robot-mounted anchor 1545, anchor 1540, and any mobile units 1510. The anchor 1540 transmits this data to server 1530 and then the anchor 1545 reenters the ACTIVE MODE and the process repeats.

As shown in FIG. 17E, at power on, mobile unit 1510 enters ACTIVE MODE and proceeds to scan for nearby active mobile units 1510, anchors 1540, and robot-mounted anchors 1545.

After scanning, the mobile unit 1510 compiles a list of SEEN mobile units 1510, anchors 1540, and robot-mounted anchors 1545 that were obtained running the scan and filters the results to find the closest anchor 1540.

The mobile unit 1510 then determines whether there is an anchor 40 nearby.

If there is no anchor 1540 nearby, then the mobile unit 1510 enters STANDBY MODE, adjusts the SLEEP DURATION INTERVAL, enters SLEEP MODE, and then waits for the SLEEP DURATION INTERVAL before re-entering ACTIVE MODE and repeating the process.

If there is an anchor 1540 nearby, then the mobile unit 1510 determines whether the anchor 1540 is within the SCAN RANGE.

If the anchor 1540 is not within the SCAN RANGE, then the mobile unit 1510 performs the scan for any nearby mobile units 1510, anchors 1540, and robot-mounted anchors 1545 step and repeats the process.

If the anchor 1540 is within the SCAN RANGE, then the mobile unit 1510 calculates the distance between the mobile units 1510, anchors 1540, and the robot-mounted anchors 1545.

The mobile unit 1510 then determines whether an access point 1520 is within communication range.

If an access point 1520 is not within the communication range, then the mobile unit 1510 reenters the ACTIVE MODE and repeats the process.

If an access point 1520 is within the communication range, then the mobile unit 1510 sends the data to the server 1530 via access point 1520 and then performs the scan for any nearby mobile units 1510, anchors 1540, and robot-mounted anchors 1545 step and repeats the process.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the methods and embodiments described herein. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc, laser disc, optical disc, digital versatile disc, floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the present subject matter. Various modifications to these embodiments will be readily apparent to those skilled in the art, and the generic principles defined herein may be applied to other embodiments without departing from the spirit or scope of the subject matter. Thus, the present subject matter is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the claims in the non-provisional application claiming priority to this provisional application.

### Non-Limiting Definitions

Definitions of one or more terms that will be used in this disclosure are described below without limitations. For a person skilled in the art, it is understood that the definitions are provided just for the sake of clarity and are intended to include more examples than just provided in the detailed description.

“ACTIVE MODE” is when the device (e.g., mobile unit 10) begins scanning for other devices. After each scan, the device software can loop through the SEEN devices and categorize them such as by safety/contact level (e.g., “SAFE”, “PRELIMINARY CONTACT”, or “CONTACT”).

“ACTIVE MODE: STATUS INDICATOR” is the alert provided by the device (e.g., mobile unit 10) after scanning and categorizing SEEN devices. The device will only activate one alert at a time. Exemplary alerts include NOK (NOT OK): RED LED and haptic feedback; WARNING: Yellow LED and haptic feedback; and OK: green LED.

“BATTERY INDICATION MODE” is when the device indicates the amount of remaining battery life, such as by LED indication, and can occur as a result of user instruction such as double tap.

“COMPARISON LOOP” is loop through the logic to do an event comparison to every other mobile unit within scanning distance from the current mobile unit.

“CONTACT DURATION”, optionally in seconds, is cumulative amount of time two or more mobile units are within the distance threshold from each other.

“CONTACT EVENT” is an occurrence where two or more mobile units have been classified as being within the distance threshold longer than the duration threshold. This term may also be defined by the system needs. For example, contact events could be defined as time spent in a specific location or within a distance of an anchor.

“DISTANCE THRESHOLD” is the maximum distance two or more mobile units may be before an interaction is considered a preliminary contact event.

“DURATION THRESHOLD” is the maximum amount of time two or more mobile units may be within the distance threshold before the interaction is considered a contact event.

“EVENT DETERMINATION” is the categorization for each event based on, for example, whether SEEN FOBS are within a DISTANCE THRESHOLD, and activity after a Preliminary Contact Event and CONTACT EVENT.

“EVENT LOG” is the data that is recorded for preliminary contact events and contact events.

“CHARGING MODE” is when the device (e.g., mobile unit 10) is in a charging mode and does not scan for other devices. The device can still perform WI-FI UPLINK.

“NOK ALERT” is the settings to alert the wearer/others around that the wearer has entered into an NOK state (i.e. a contact event).

“OK ALERT” is the settings to alert the wearer/others around that the wearer has entered into an OK state (i.e. ended a contact event).

“PRELIMINARY CONTACT EVENT” is an occurrence where two or more mobile units are within the distance threshold for less than the duration threshold.

“SCAN ACTIVE SETPOINT” is the setpoint for the scan interval at which to passively scan for other mobile units.

“SCAN RANGE” is the maximum distance to actively search for other mobile units.

“SCAN SETTINGS” are the settings for each scan and include SCAN RANGE and STANDBY THRESHOLD.

“SEEN FOB” is another mobile unit that has been seen by this mobile unit during the last scan interval and denotes which mobile unit is active in the comparison loop.

“SEPARATION DISTANCE” is the minimum distance between two or more mobile units before an interaction is no longer considered a contact event (i.e. distance is past the distance threshold hysteresis).

“SLEEP DURATION INTERVAL” is the amount of time to remain in SLEEP MODE (low energy/battery saving state) before scanning for other mobile units.

“SLEEP MODE” is when the device (e.g., mobile unit 10) enters a battery saving state for a period of time (e.g., SLEEP DURATION INTERVAL) and reduces the frequency of scans for other devices.

“STANDBY MODE” is when the device (e.g., mobile unit 10) adjusts the parameters (e.g., SLEEP DURATION INTERVAL) to enter SLEEP MODE.

“STANDBY THRESHOLD” is the maximum amount of time for the mobile unit to be in active mode without seeing another mobile unit within the scan range.

“TRANSMISSION INTERVAL” is the interval in which the current mobile unit connects to the network to receive any updates from the reporting server or to submit event logs.

“WARNING ALERT” is the settings to alert the wearer/others around that the wearer has entered into a warning state (i.e. a preliminary contact event).

“WI-FI UPLINK” is when the device (e.g., mobile unit 10) periodically connects, according to the TRANSMISSION INTERVAL, to the network (e.g., via access point 20, to upload and/or download data from the server (e.g., server 30).

What is claimed is:

1. A tracking system comprising:
a plurality of mobile units which are capable of communication with a server over a wireless network;
wherein the plurality of mobile units are configured to determine whether the mobile unit is in a contact event when a first mobile unit is within a predetermined distance from a second mobile unit for a predetermined amount of time using ultra-wideband positioning;
wherein the first mobile unit reports to the server if the first mobile unit is in a contact event.

2. The tracking system of claim 1 further comprising:
an access point capable of communication with the server over the wireless network, wherein
the plurality of mobile units are configured to communicate with the server via the access point.

3. The tracking system of claim 1, wherein
the mobile units comprises a first mobile unit and a second mobile unit, and
the first mobile unit is configured to determine the presence of a preliminary contact event when the first mobile unit is within the predetermined distance from the second mobile unit for less than a predetermined amount of time.
